(19) United States
(12) Patent Application Publication
(10) Pub. No.: US 2021/0287763 A1
(43) Pub. Date: Sep. 16, 2021

(54) SYSTEM AND METHOD FOR SELECTING A SET OF CANDIDATE DRUG COMPOUNDS

(71) Applicant: Innoplexus AG, Eschborn (DE)

(72) Inventor: Om Sharma, Pune (IN)

(73) Assignee: Innoplexus AG, Eschborn (DE)

(21) Appl. No.: 17/202,931

(22) Filed: Mar. 16, 2021

Related U.S. Application Data
(60) Provisional application No. 62/990,117, filed on Mar. 16, 2020.
(60) Provisional application No. 62/990,125, filed on Mar. 16, 2020.
(60) Provisional application No. 62/990,129, filed on Mar. 16, 2020.

(51) Int. Cl. G16C 20/50; G16B 15/30; G16B 5/20

(52) U.S. Cl. CPC G16C 20/50; G16B 5/20; G16B 15/30

(57) ABSTRACT

A method for selection of a set of candidate drug compounds includes generating a plurality of knowledge-based pathways based on at least relevant information. The relevant information is extracted from structured information based on an ontology of interest. A set of target structures is identified based on the plurality of knowledge-based pathways. A plurality of candidate drug compounds is determined for the identified set of target structures. Based on safety analysis of the plurality of candidate drug compounds using a lethality index, a set of candidate drug compounds is selected from the plurality of candidate drug compounds.

### CROSS-REFERENCE TO RELATED APPLICATIONS/INCORPORATION BY REFERENCE

This Patent Application claims priority to, and the benefit from United States Provisional Application Ser. No. U.S. 62/990,117, U.S. 62/990,125, and U.S. 62/990,129, filed Mar. 16, 2020.

Each of the above referenced patent applications is hereby incorporated herein by reference in its entirety.

### FIELD OF TECHNOLOGY

Certain embodiments of the disclosure relate to a method and system for repurposing drug compounds. More specifically, certain embodiments of the disclosure relate to a method and system for selection of a set of candidate drug compounds.

### BACKGROUND

Despite advances in technology and enhanced understanding of biological systems, drug discovery is still a lengthy, expensive, difficult, and inefficient process with a low rate of new therapeutic discovery. Therefore, for decades, researchers, scientists, and academic institutions have been advocating the idea of screening libraries of existing approved drugs compounds to identify or uncover new indications, which is termed as drug repurposing. Because the safety of these drugs has already been tested in clinical trials for other applications, repurposing known drug compounds may treat emerging and challenging diseases, including COVID-19, much faster and with less cost than that of developing new drugs.

To uncover the potential of drug repurposing, various technologies are being leveraged. However, the systems and/or method of such technologies are struggling to shortlist appropriate candidate drug compounds, and also identify target structures with the least error. This may cause hindrance in the therapeutic development of emerging and challenging diseases in medical emergency situations, such as an epidemic or pandemic.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY OF THE DISCLOSURE

Systems and/or methods are provided for selection of a set of candidate drug compounds, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram that illustrates an exemplary system for selection of a set of candidate drug compounds, in accordance with an exemplary embodiment of the disclosure.

FIG. 2 illustrates an exemplary schematic representation depicting a knowledge-based graphical network for a plurality of knowledge-based pathways, in accordance with an exemplary embodiment of the disclosure.

FIG. 3 illustrates an exemplary schematic representation of molecular interactions in a biological network, in accordance with an exemplary embodiment of the disclosure.

FIGS. 4A and 4B illustrate two exemplary schematic representations of protein-protein interaction (PPI) network cluster between molecular interactions in the biological network, in accordance with an exemplary embodiment of the disclosure.

FIGS. 5A and 5B depict flowchart illustrating exemplary operations for selection of a set of candidate drug compounds, in accordance with an exemplary embodiment of the disclosure.

FIG. 6 is a conceptual diagram illustrating an example of a hardware implementation for a system employing a processing system for selection of a set of candidate drug compounds, in accordance with an exemplary embodiment of the disclosure.

### DETAILED DESCRIPTION OF THE DISCLOSURE

Certain embodiments of the disclosure may be found in a method and system for selection of a set of candidate drug compounds. Various embodiments of the disclosure provide a method and system that correspond to AI-driven drug discovery engine powered by proprietary life science repository that can efficiently identify the target structure, mechanism of action (MOA), knowledge-based pathway and candidate drug compounds for given indication in minimal response time. The proposed method and system may be configured to precisely select candidate drug compounds, and combinations of candidate drug compounds for drug repurposing. Such combinations of candidate drug compounds are thoughtfully placed together considering their MOAs, knowledge-based pathways, biological processes and safety profiles.

In accordance with various embodiments of the disclosure, a method may be provided for selection of a set of candidate drug compounds. The method may include generating, by one or more processors, a plurality of knowledge-based pathways based on at least relevant information. The relevant information may be extracted from structured information based on an ontology of interest. The method may further include identifying a set of target structures based on the plurality of knowledge-based pathways, determining a plurality of candidate drug compounds for the identified set of target structures, and selecting a set of candidate drug compounds from the plurality of candidate drug compounds based on safety analysis of the plurality of candidate drug compounds using a lethality index. The lethality index corresponds to a scatter plot with safety coordinates which positions adverse events on a universal lethality index versus a universal frequency index

In accordance with an embodiment, the ontology of interest may be a life science ontology that comprises a plurality of biomedical terms and a plurality of data connections. The structured information comprises at least a number of principal investigators, intervention used in clinical trials, expressions, biological functions, mutations and mechanism of actions retrieved from the relevant publications and the clinical trial registries associated with a medical condition of the host entity.

In accordance with an embodiment, the method may include retrieving, by the one or more processors, unstructured data from data sources via interfaces and application program interfaces (APIs). The data sources store a repository of publications, clinical trials, congresses, patents, grants, drug profiles, and gene profiles.

In accordance with an embodiment, the method may include extracting, by the one or more processors, the structured information from the unstructured data based on one or more artificial intelligence and natural language processing techniques.

In accordance with an embodiment, the method may include performing, by the one or more processors, a computational docking-based virtual screening for prioritization of a first set of candidate drug compounds corresponding to the identified set of target structures based on one or more scores. The plurality of candidate drug compounds may be determined based on the first set of candidate drug compounds. A first score of the one or more scores may be a quantitative docking score that corresponds to performance of each candidate drug compound for each target structure. A second score of the one or more scores may be an affinity score that corresponds to an overall strength of binding affinity of each candidate drug compound based on a spatial arrangement of docking pose and presence of hydrogen bond interactions with each target structure.

In accordance with an embodiment, the method may include determining, by the one or more processors, a second set of candidate drug compounds based on a plurality of direct and in-direct connections between a plurality of biological entities in a biological network and the ontology of interest. The plurality of candidate drug compounds may be determined based on the second set of candidate drug compounds.

In accordance with an embodiment, the method may include determining, by one or more processors, a third set of candidate drug compounds based on a first analysis and a second analysis. The first analysis may be associated with the gene and protein expression profile of the identified set of target structures. The second analysis may be associated with expression profiles of the third set of candidate drug compounds and corresponding pharmacokinetics effect. The plurality of candidate drug compounds may be determined based on the third set of candidate drug compounds.

In accordance with an embodiment, the method may include normalizing, by the one or more processors, the plurality of candidate drug compounds based on cross-mapping through the ontology of interest.

In accordance with an embodiment, the method may include scoring, by the one or more processors, the plurality of candidate drug compounds based on one or more parameters.

In accordance with an embodiment, the method may include performing, by one or more processors, molecular dynamics simulation on the plurality of candidate drug compounds to identify interaction stability with the identified set of target structures.

In accordance with an embodiment, the method is provided for determining a combination of candidate drug compounds. The method may include determining, by one or more processors, prioritized target structures based on mapping of a set of target structures and a list of target structures. The method may further include identifying a plurality of data connections, corresponding to the prioritized target structures, from the plurality of biological networks, and determining a target connection network corresponding to the identified plurality of data connections. The method may further include detecting a plurality of clusters corresponding to the plurality of data connections in the target connection network based on a graph-embedded self-clustering technique, and determining at least a first drug combination of at least a first candidate drug compound and a second candidate drug compound based on a combination score. The first candidate drug compound corresponds to a first cluster and the second candidate drug compound corresponds to a second cluster.

In accordance with an embodiment, the method may include identifying, by one or more processors, the set of target structures corresponding to a set of candidate drug compounds. In accordance with an embodiment, the method may include identifying, by one or more processors, a gene ontology corresponding to a host viral interaction and an associated list of target structures. The set of candidate drug compounds may be selected from a plurality of candidate drug compounds based on safety analysis of the plurality of candidate drug compounds using a lethality index.

In accordance with an embodiment, the method may include mapping, by the one or more processors, each of the set of candidate drug compounds with a target structure of each cluster.

In accordance with an embodiment, the method may include calculating, by the one or more processors, the combination score for at least the first drug combination based on at least docking scores, lethality scores and safety scores corresponding to the first candidate drug compound and the second candidate drug compound. The combination score for at least the first drug combination may exceed a threshold value.

In accordance with an embodiment, the method may include determining, by the one or more processors, a rank of the first drug combination based on a corresponding percentile score with respect to other drug combinations.

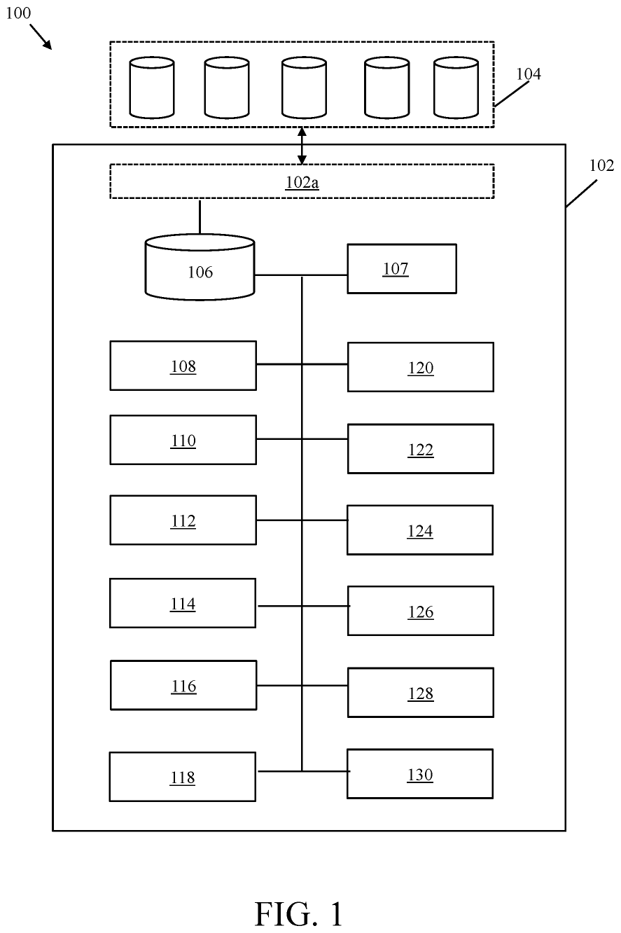

FIG. 1 is a block diagram that illustrates an exemplary system for selection of a set of candidate drug compounds, in accordance with an exemplary embodiment of the disclosure. Referring to FIG. 1, a computing environment 100 that includes at least a system 102 and data sources 104 external to the system 102. The system 102 comprises a set of interfaces 102a, a knowledge base 106, knowledge processing engines 107, and a set of ontologies 108. The system 102 further comprises a pathway generation engine 110, a search engine 112, a screening engine 114 an expression analysis engine 116, an aggregation, normalization and scoring (ANS) engine 118, a molecular stability analysis engine 120, and a safety analysis engine 122. The system 102 further comprises a network analysis engine 124, a clustering engine 126, a drug selection engine 128, and a user interface 130.

In some embodiments of the disclosure, one or more processors, such as the knowledge processing engines 107 may be integrated with other engines to form an integrated system. In some embodiments of the disclosure, as shown, the knowledge processing engines 107 may be distinct from the other engines. Other separation and/or combination of the various processing engines and entities of the exemplary system 102 illustrated in FIG. 1 may be done without departing from the spirit and scope of the various embodiments of the disclosure.

Without any deviation from the scope of the disclosure, one or more processors described herein, such as the knowledge processing engines 107, the pathway generation engine 110, the search engine 112, the screening engine 114, the expression analysis engine 116, the ANS engine 118, the molecular stability analysis engine 120, the safety analysis engine 122, the network analysis engine 124, the clustering engine 126, and the drug selection engine 128 may be collectively referred to as ‘drug discovery engine’.

The data sources 104 may correspond to a plurality of resources, such as servers and machines, that may store a repository of publications, clinical trials, congresses, patents, grants, drug profiles, gene profiles, and the like. Such data sources 104 may comprise unstructured and disparate data having variable structures. The unstructured data may be retrieved from the data sources 104 via various interfaces and application program interfaces (APIs), such as the set of interfaces 102a in the system 102. The set of interfaces 102a in the system 102 may be configured to convert the unstructured data into such a format that may be appropriately handled by the knowledge processing engines 107 to store in the knowledge base 106.

The unstructured data may be digitized information that is available in a non-formalized structure, which is not relational and is not organized in a uniform, pre-defined traditional row-column database. Such unstructured data may include, for example text like eMail messages, service-center transcripts, powerpoint presentations, survey responses, news, research papers, scientific posters, patent data, patient medical records, authors names, webpages, PDF files, journals, documents, metadata, social media forums, posts, tweets, blogs, images like pdf, graphs, photos, x-rays/MRIs, audio files, recorded voice, music, video, machine data, log files, and sensor data.

The knowledge base 106 may comprise suitable logic, circuitry, and interfaces that may be configured to execute code that may store structured information extracted from the unstructured data based on an ontology of interest from the set of ontologies 108, such as life sciences ontology. The extraction may be based on one or more artificial intelligence (AI) powered and natural language processing (NLP) techniques that may be executed by the knowledge processing engines 107.

The knowledge processing engines 107 may comprise suitable logic, circuitry, and interfaces that may be configured to execute code that may perform a plurality of functionalities, in conjunction with other processors (or engines), based on one or more of the AI, NLP, and machine learning (ML) techniques. In accordance with an embodiment, the knowledge processing engines 107 may be configured to extract the structured information from the unstructured data.

In accordance with certain embodiments, to generate the structured information from the unstructured data, the knowledge processing engines 107 may extract meta-data from content, such as concepts, entities, keywords, categories, sentiment, emotion, relations, semantic roles, and the like, based on natural language understanding. Further, deep learning algorithms in the knowledge processing engines 107 may utilize neural networks to analyze the unstructured data seeking to understand complex problems, such as interpreting images or text-based natural language and human speech. In accordance with other embodiments, the knowledge processing engines 107 may execute speech recognition algorithms, computer vision and image recognition algorithms to extract the structured information from unstructured audio data, pdf files, and video data, respectively.

The structured information, thus generated, may include, but not limited to, a number of principal investigators, intervention used in clinical trials, expressions, biological functions, mutations and mechanism of actions retrieved from the relevant publications and the clinical trial registries associated with a medical condition of a host entity. The structured information may further contain information about authors, researchers, hospitals, regulatory body decisions, health technology assessment (HTA) body decisions, treatment guidelines, biological databases of genes, proteins, and pathways, patient advocacy groups, patient forums, social media posts, news, and blogs.

In accordance with an embodiment, the knowledge processing engine 107 may be further configured to utilize the linguistic, auditory, and visual structure that exists in all forms of human communication to generate the structured information. In accordance with an embodiment, the knowledge processing engines 107 may be configured to deploy text analytics tools that may be configured to identify patterns, keywords, and sentiment in textual data by examining word morphology, sentence syntax, as well as other small-scale and large-scale patterns.

In accordance with an embodiment, the knowledge processing engine 107 may be configured to extract relevant information from the structured information based on an ontology of interest. Thus, the relevant information may correspond to a subset of the structured data, such as the number of principal investigators, intervention used in clinical trials, expressions, biological functions, mutations and mechanism of actions retrieved from the relevant publications and the clinical trial registries associated with COVID-19, that correspond to the ontology of interest.

In accordance with an embodiment, the knowledge processing engines 107, in conjunction with the search engine 112, may be configured to determine a second set of candidate drug compounds. The second set of candidate drug compounds may be non-obvious potential candidate drug compounds for the set of target structures. The knowledge processing engines 107 may leverage the search engine 112, i.e. Ontosight Explore®, which is an ontology-based biological network of protein, pathways, drugs and diseases to determine a second set of candidate drug compounds.

The set of ontologies 108 may correspond to automated self-updating databases of data sets (encompassing domain-specific terms and synonyms), semantic associations, and concepts of a specific domain, such as life sciences, biomedical, or genomes. Using machine learning (ML) algorithms, the ontology of interest from the set of ontologies 108 may add new terms and connections to the knowledge base 106. The set of ontologies 108 may provide recommendations for missing side effects, warnings, and the like through sentiment analysis on reviews. The set of ontologies 108 may facilitate in segregating the extracted structured information or unstructured data, and enable the one or more processors to focus on most relevant ontology-specific content. In an exemplary scenario, a life sciences ontology facilitates the search engine 112 to establish relationships between biological entities, such as genes, proteins, diseases, and drugs, as well as helps in discovering new connections.

In accordance with an embodiment, the set of ontologies 108 may be generated in conjunction with the knowledge processing engines 107 that may be configured to crawl, aggregate, analyze semantic associations, and visualize the unstructured and structured information based on a search query. The crawling may be done through the unstructured data and structured information. The crawled data may be validated based on one or both of an automated as well as manual validation process. Afterwards, the validated data may be normalized and aggregated into relevant data sets, which is machine-readable, and in a structured form. The normalized data may be then analyzed for patterns, relations, entities, and semantic associations. The results, that are validated and accurate, may be presented in an intuitive interface with visualizations to generate the most relevant insights to be stored in the knowledge base 106 in real-time.

In accordance with an embodiment, each of the set of ontologies 108 may map discoverable concepts from all major sources, connect observations, and learn unseen concepts. This may help researchers, academicians, and scientists to generate associations between disease, gene, drug compounds, target structures, molecules, MOAs, and the like. Further, a search performed using specific concepts and terms in the ontology of interest (instead of tagged words) may help minimize manual intervention and automate identification and tagging of the most relevant content.

The pathway generation engine 110 may comprise suitable logic, circuitry, and interfaces that may be configured to execute code that generates a plurality of knowledge-based pathways based on at least the structured information retrieved from the unstructured data using an ontology of interest, such as life science ontology. In accordance with an embodiment, the pathway generation engine 110, in conjunction with the knowledge processing engines 107, may be configured to generate the plurality of the knowledge-based pathways.

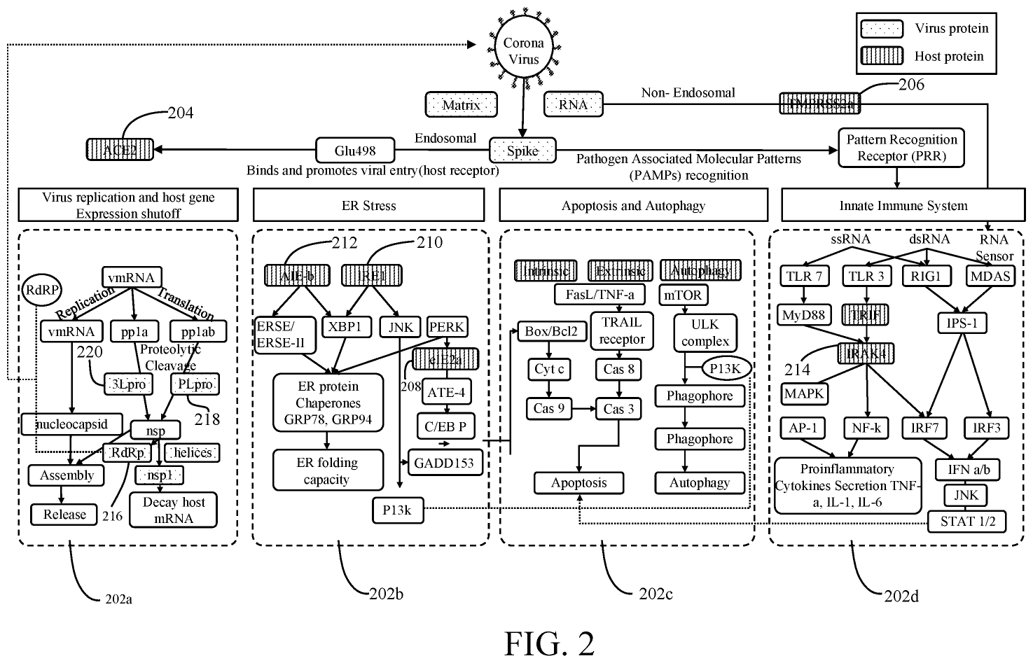

In accordance with the exemplary embodiment, the pathway generation engine 110 may be configured to generate a knowledge-based graphical network based on information of host factors co-opted during individual stages of infection replication. The knowledge-based graphical network may include a plurality of knowledge-based pathways generated based on information of signaling pathways activated during an infection, stress response, autophagy, apoptosis, and innate immunity, as described in detail in FIG. 2.

In accordance with an embodiment, the pathway generation engine 110 may be further configured to identify a set of target structures based on the plurality of knowledge-based pathways, as described in FIG. 2. For example, the identified set of target structures may correspond to the host protein and the virus protein in case of COVID-19 infection. Examples of the set of target structures may include, for example, angiotensin-converting enzyme-2 (ACE2) 204, Transmembrane Protease Serine-2 (TMPRSS2) 206, Eukaryotic Initiation Factor 2 alpha (eIF2α) 208, Inositol-requiring enzyme-1 (IRE1) 210, Activating Transcription Factor-6 (ATF6) 212, interleukin-1 receptor-associated kinase 4 (IRAK4) 214, RNA-dependent RNA polymerase (RdRp) 216, and papain-like protease (PLpro) 218 and the 3C-like protease (3CLpro) 220, as illustrated in FIG. 2.

The search engine 112 may comprise suitable logic, circuitry, and interfaces that may be configured to execute code that may be configured to determine the second set of candidate drug compounds. Such plurality of candidate drug compounds may be non-obvious therapeutic interventions that may be ranked based on association score and grouped based on the set of target structures for the disease. An example of such search engine 112 may be Ontosight□Explore.

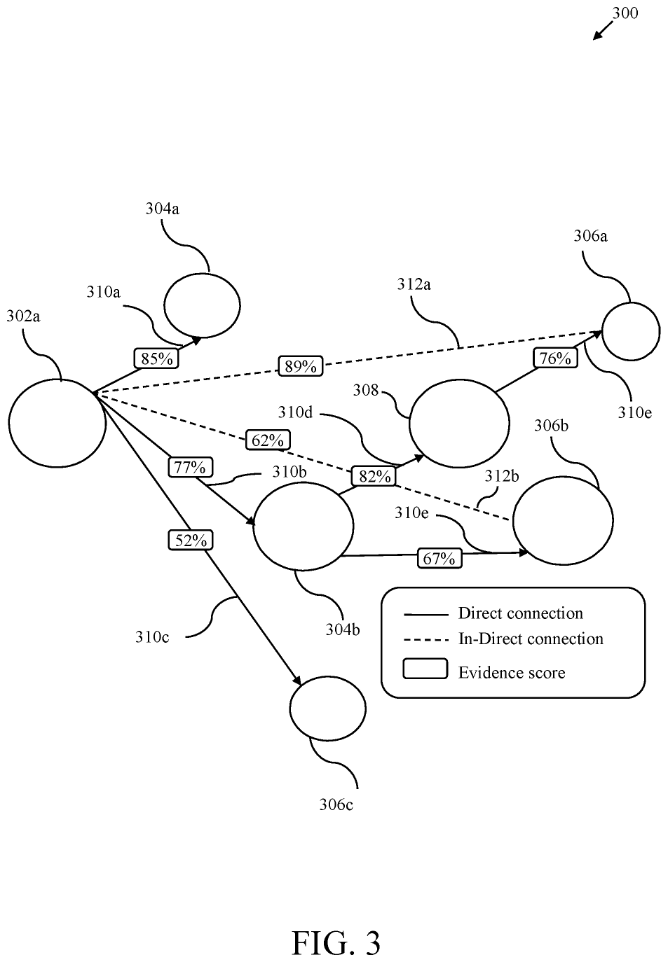

In accordance with an embodiment, the search engine 112, in conjunction with the knowledge processing engines 107, may explore and identify obvious and non-obvious molecular interconnections, interchangeably referred to as ‘data connections’, between diseases, knowledge-based pathways, proteins/target structures, and a plurality of candidate drug compounds within a biological network in accordance with an ontology of interest, based on one or more AI and NLP techniques. The search engine 112 may indicate interconnectedness of the biological networks with regard to corresponding search terms, which may be a gene, a target structure/protein, a knowledge-based pathway, or a disease. The search engine 112 may aggregate all of the set of target structures, a library of drug compounds, diseases with associated known and potential plurality of knowledge-based pathways and a series of molecular interactions which are responsible for its origin and severity, as illustrated in FIG. 3.

In accordance with an embodiment, the search engine 112 may further identify alternative indications for given drug compounds through indirectly associated indications through alternative target structures and knowledge-based pathways. The search engine 112 may further rank such associations and prioritize assets based on commonality/association, druggability and druglikeness.

In accordance with another embodiment, the search engine 112 may be configured to identify a list of target structures for the set of candidate drug compounds based on an ontology of interest from the set of ontologies 108, such as gene ontology. In such an embodiment, the gene ontology may be an automated self-updating database of data sets (encompassing genomic terms and synonyms), semantic associations, and concepts of genomes. Examples of such concepts associated with host-viral interaction may include, but are not limited to, endocytosis involved in viral entry into host cell (GO:0075509), suppression by virus of host adaptive immune response (GO:0039504), modulation by virus of host protein ubiquitination (GO:0039648), positive regulation by symbiont of host receptor-mediated endocytosis (GO:0044078), and ubiquitin-dependent protein catabolic process (GO:0006511).

The screening engine 114 may comprise suitable logic, circuitry, and interfaces that may be configured to execute code that prioritizes a first set of candidate drug compounds for the identified set of target structures based on one or more scores. The screening engine 114 may perform a computational docking-based virtual screening for the prioritization of the plurality of candidate drug compounds corresponding to the identified set of target structures. A first score of the one or more scores may be a quantitative docking score that corresponds to performance of each candidate drug compound for each target structure. A second score of the one or more scores may be an affinity score that corresponds to an overall strength of binding affinity of each candidate drug compound based on a spatial arrangement of docking pose and presence of hydrogen bond interactions with each target structure.

In accordance with an embodiment, three-dimensional (3D) structures of each of the set of target structures may be retrieved from a protein data bank (PDB). In case of multiple crystal entries for a given target structure, preference may be given to a structure entry where a drug-like molecule is co-crystallized and a good resolution of structure entry is available. For virtual screening, the protein files may prepared for an automated tool, such as AutoDockTools®, by removing cocrystal ligands and water molecules from the 3D structure, adding hydrogen atoms and partial charges (Gasteiger), and saving coordinates of the 3D structures in a specified format, such as pdbqt format, for further molecular docking process. Grid of the proteins may be generated by using the cocrystal ligands as the reference. In an exemplary scenario, the 3D structure of top candidate drug compounds for identified proteins may be downloaded from PubChem® and the structure may be minimized & converted to pdb format using a chemical toolbox, such as Open babel®. For visualization of docked poses, an interactive visualization tool, such as UCSF Chimera may be used. Thereafter, an open-source program, such as Autodock vina 1.1.2, may be used to perform the docking based virtual screening of the plurality of candidate drug compounds against the X-Ray structure of the set of target structures. For preparation of protein receptors and screening chemical libraries, AutoDockTools® may be used. The set of target structures may be loaded individually, Hydrogens, and thereafter Gasteiger charges may be added. Unwanted crystal adducts may be deleted and a pdbqt file may be saved. The bound crystal ligand of individual target structure may be used as a reference for the selection of binding sites. AutoDockTools® may also be used for the energy minimization of drug compounds and for converting all molecules to AutoDock Ligand format (PDBQT). Standard grid may be generated for each of the set of target structures based on their critical binding residues. The screening engine 114 may perform virtual screening in a high-performance computing environment and prioritize the plurality of candidate drug compounds for the identified set of target structures based on the one or more scores.

The expression analysis engine 116 may comprise suitable logic, circuitry, and interfaces that may be configured to execute code that may determine a third set of candidate drug compounds based on a first analysis and a second analysis. The first analysis may be associated with the gene and protein expression profile of the identified set of target structures. The second analysis may be associated with expression profiles of the third set of candidate drug compounds and corresponding pharmacokinetics effect. In an exemplary embodiment, the expression analysis engine 116 may perform the first and second analysis based on literature mining. The expression analysis engine 116 may perform ontology-based search in the unstructured data for specific drug modulation(s) in the identified set of target structures, for example drug ‘x’ up-regulate or downregulate the ‘Y’ target structures in a Covid-19 patient. In an exemplary embodiment, the expression analysis engine 116 may perform the first and second analysis based on extraction of similar disease sample, such as SARS CoV, MERS, and the like, for a target disease, such as Covid-19, and identify treated drug compound(s) and corresponding responder genes/proteins.

The ANS engine 118 may comprise suitable logic, circuitry, and interfaces that may be configured to execute code that determines a plurality of candidate drug compounds for the identified set of target structures based on the first, the second and the third set of candidate drug compounds. The ANS engine 118 may aggregate the first, the second and the third set of candidate drug compounds identified from the screening engine 114, the search engine 112, and the expression analysis engine 116, respectively, and generate a normalized unique list of drug compounds by cross-mapping through the ontology of interest from the set of ontologies 108. The ANS engine 118 may further perform scoring of the normalized unique list of drug compounds based on one or more of the clinical trials for a specific disease, such a Covid-19 (Exists—0/No Exists—1), a safety score of a drug compound (Tolerable Adverse events—1, Severe adverse events—0), expression profiles (Drug respond to the identified set of target structures?), approved drug compound (Other indication) or novel drug compounds (Approved—1, Novel—0, Clinical drug—1), patent evidence for drug repurposing (No—1, Yes—0), literature evidences for any COVID-19 similar virus (Yes—1, No—0), and cumulative scores of above mentioned evaluation parameters.

The molecular stability analysis engine 120 may comprise suitable logic, circuitry, and interfaces that may be configured to execute code that performs molecular dynamics simulation (MDS) study for top drug compounds to identify their interaction stability with identified proteins. The most stable proteins and drug compound combinations may be selected based on protein-ligand complex root-mean-square deviation (RMSD) values.

The safety analysis engine 122 may comprise suitable logic, circuitry, and interfaces that may be configured to execute code that may select a set of candidate drug compounds from the plurality of candidate drug compounds based on safety analysis of the plurality of candidate drug compounds using a lethality index. The safety analysis engine 122 may perform the safety analysis using an adverse event analysis protocol, such as lethality index. In accordance with an embodiment, the lethality index is a scatter plot with safety coordinates which efficiently positions adverse events on the ‘X’ and ‘Y’ axis, such as universal lethality index (ULI) versus universal frequency index (UFI) respectively. The ULI and UFI may be calculated based on publicly available adverse events, severity, frequency and outcome within a specific time frame. Mathematically, the safety coordinates, UFI and ULI may be expressed as following equation (1):

$$Safety Coordinates = ( {ULI}_{E} , {UFI}_{E} )$$

$${UFI}_{E} = \frac{\vert D ⋂ {D}_{E} \vert}{\vert D \vert} and ULI = \frac{4}{\vert {F}_{E} \vert} {\sum }_{i = 1}^{\vert {F}_{E} \vert} ( {F}_{E}^{i} \times {Q}_{E}^{i} )$$

$$Further , \\ {F}_{E}^{i} = \frac{\vert {FR}_{E}^{i} ⋂ {R}_{E}^{i} \vert}{\vert {R}_{E}^{i} \vert} and {Q}_{E}^{i} = \{ 1 , {F}_{E}^{i} ∈ {Q}_{4} ( {F}_{E} ) 0 , {F}_{E}^{i} ∉ {Q}_{4} ( {F}_{E} )$$

where D={d: all drug compounds d with reported adverse events in public databases},
D_{E}={d: all drug compounds d with reported adverse event E},
R_{E}ⁱ=reports at time interval T,
FR_{E}ⁱ=fatal reports at time interval T,
F_{E}=F_{E}ⁱ all time intervals T, and
Q₄=Upper quartile.

The network analysis engine 124 may comprise suitable logic, circuitry, and interfaces that may be configured to execute code that identifies a plurality of data connections corresponding to prioritized target structures from the plurality of biological networks. Such data connections may correspond to molecular interactions between each of the identified prioritized target structures and other biological entities, such as candidate drug compounds. In accordance with an embodiment, the network analysis engine 124 may be configured to determine a target connection network corresponding to the identified plurality of data connections.

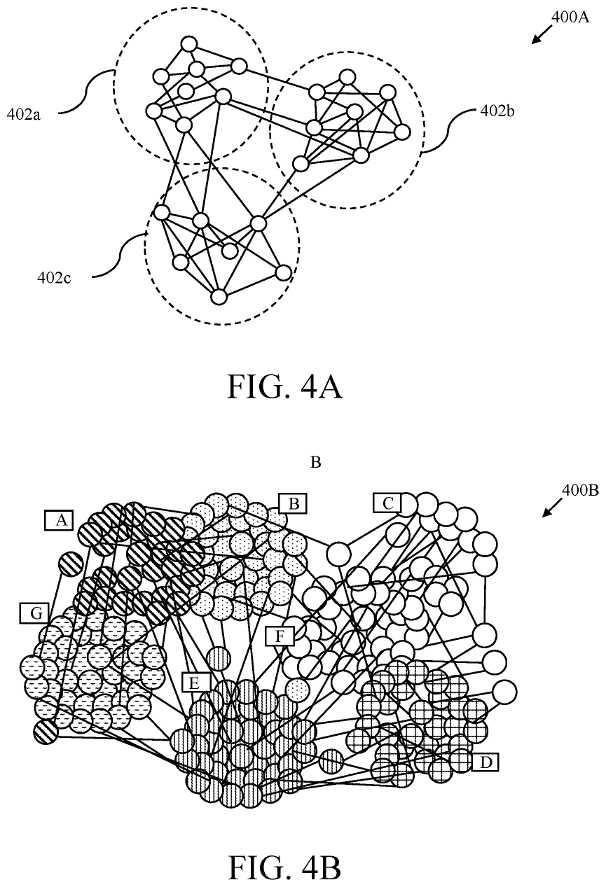

The clustering engine 126 may comprise suitable logic, circuitry, and interfaces that may be configured to execute code that detects a plurality of clusters corresponding to the plurality of data connections in the target connection network based on graph-embedded self-clustering technique. In accordance with the graph-embedded self-clustering technique, the clustering engine 126 may iteratively embed nodes with neighbor nodes in the target connection network, and detect the clusters. The graph-embedded self-clustering technique may use a paradigm of sequence-based node embedding procedures that may create ‘d’ dimensional feature representations of nodes in an abstract feature space. Sequence-based node embeddings may embed pairs of nodes close to each other if they occur frequently within a small window of each other in a random walk and minimize the negative log-likelihood of observed neighborhood samples. An exemplary set of clusters and corresponding clusters rendered in D3 force graphs are illustrated in FIGS. 4A and 4B respectively.

The drug selection engine 128 may comprise suitable logic, circuitry, and interfaces that may be configured to execute code that performs mapping of each of the set of candidate drug compounds with a target structure of each cluster.

In accordance with an embodiment, the drug selection engine 128 may determine at least a first drug combination of at least a first candidate drug compound and a second candidate drug compound based on a combination score. The first candidate drug compound corresponds to a first cluster and the second candidate drug compound corresponds to a second cluster. The drug selection engine 128 may perform multiple permutation and combination in a group of two candidate drug compounds. Such multiple permutation and combination may be generated such that both drug compounds of the combination should correspond to at least two different clusters. It may be noted that the majority of the candidate drug compounds correspond to different clusters while some candidate drug compounds may be associated with more than one cluster group target structures based on the random walk and neighbor likelihood score.

In accordance with an embodiment, the drug selection engine 128 may be configured to calculate a combination score for at least the first drug combination based on at least docking scores, lethality scores and safety scores corresponding to the first candidate drug compound and the second candidate drug compound. The combination score for at least the first drug combination exceeds a threshold value. Mathematically, the combination score may be expressed as the following equation (2):

$$C = \frac{\sum ( {D}_{{S}_{D 1}} , {D}_{{S}_{Dn}} ) / N}{\sum ( {L}_{D 1} , {L}_{Dn} ) / \sum ( {S}_{D 1} , {S}_{Dn} )}$$

where C=Combination score,
D=candidate drug compound,
Ds=Docking score,
N=n number of candidate drug compounds used in combination,
L=Lethality score, and
S=Safety score.

In accordance with an embodiment, the drug selection engine 128 may be configured to determine a rank of the first drug combination based on a corresponding percentile score with respect to other drug combinations. The percentile score may be calculated for each drug combination. The calculation of the percentile may be performed based on generic percentile calculation methods known in the art.

The user interface 130 may comprise suitable logic, circuitry, and interfaces that may be configured to present the results of the safety analysis engine 122 and the drug selection engine 128. The results may be presented in form of an audible, visual, tactile or other output to a user, such as a researcher, a scientist, a principal investigator, and a health authority, associated with the system 102. As such, the user interface 130 may include, for example, a display, one or more switches, buttons or keys (e.g., a keyboard or other function buttons), a mouse, and/or other input/output mechanisms. In an example embodiment, the user interface 130 may include a plurality of lights, a display, a speaker, a microphone, and/or the like. In some embodiments, the user interface 130 may also provide interface mechanisms that are generated on the display for facilitating user interaction. Thus, for example, the user interface 130 may be configured to provide interface consoles, web pages, web portals, drop down menus, buttons, and/or the like, and components thereof to facilitate user interaction.

FIG. 2 illustrates an exemplary schematic representation depicting a knowledge-based graphical network for a plurality of knowledge-based pathways, in accordance with an exemplary embodiment of the disclosure.

With reference to FIG. 2, there is shown a knowledge-based graphical network 200 that includes a first knowledge-based pathway 202a, a second knowledge-based pathway 202b, a third knowledge-based pathway 202c, and a fourth knowledge-based pathway 202d. The first knowledge-based pathway 202a may correspond to a schematic diagram that illustrates host factors co-opted and signaling pathways activated during a host-interaction and replication, during an infection, such as COVID-19 infection. The second knowledge-based pathway 202b may correspond to a schematic diagram that illustrates host factors co-opted and signaling pathways activated during a stress response. The third knowledge-based pathway 202c may correspond to a schematic diagram that illustrates host factors co-opted and signaling pathway activated during autophagy and apoptosis. The fourth knowledge-based pathway 202d may correspond to a schematic diagram that illustrates host factors co-opted and signaling pathway activated during innate immunity. The knowledge-based pathways illustrate various therapeutic target structures that play important roles during various stages of the infection.

FIG. 3 illustrates an exemplary schematic representation of molecular interactions in a biological network, in accordance with an exemplary embodiment of the disclosure.

With reference to FIG. 3, there is illustrated a schematic representation of molecular interactions of a biological network 300. The biological network 300 may include a plurality of nodes, such as a target structure 302a from the set of target structures, a first knowledge-based pathway 304a, a second knowledge-based pathway 304b, a first drug compound 306a, a second drug compound 306b, a third drug compound 306c, and a disease 308. The size of each node represents data availability and how well the entity is explored. The biological network 300 may further include a plurality of direct interactions, such as a first direct interaction 310a between the target structure 302a and the first knowledge-based pathway 304a, a second direct interaction 310b between the target structure 302a and the second knowledge-based pathway 304b, a third direct interaction 310c between the target structure 302a and the third drug compound 306c. The biological network 300 may further include a fourth direct interaction 310d between the second knowledge-based pathway 304b and the disease 308, a fifth direct interaction 310e between the second knowledge-based pathway 304b and the second drug compound 306b, and a sixth direct interaction 310f between the disease 308 and the first drug compound 306a. Based on the plurality of direct interactions, the biological network 300 may include a plurality of indirect interactions, such as a first indirect interaction 312a between the target structure 302a and the first drug compound 306a, and a second indirect interaction 312b between the target structure 302a and the second drug compound 306b. The search engine 112 may score the plurality of direct and indirect interactions based on a number of parameters, such as druggability, druglikeness and publicly available evidence from literature, patents, grants, thesis, news and press evidence. The score is illustrated to be labeled on each of the plurality of direct and indirect interactions in FIG. 3.

FIGS. 4A and 4B illustrates two exemplary schematic representations of PPI network clusters between molecular interactions in the biological network, in accordance with an exemplary embodiment of the disclosure.

With reference to FIG. 4A, there is illustrated a PPI network cluster 400A between molecular connections in a plurality of biological networks. In the exemplary embodiment, each instance of the plurality of biological networks may be similar to the biological network 300. As illustrated in FIG. 4A, each node circle represents a target structure/protein and dotted circle represents the clustered group, such as a first cluster 402a, a second cluster 402b, and a third cluster 402c, and each edge represents a molecular interaction between the two nodes from different clusters, such as the first cluster 402a, the second cluster 402b, and the third cluster 402c.

With reference to FIG. 4A, there is illustrated another PPI network cluster 400B. The PPI network cluster 400B illustrates different cluster groups, i.e. A, B, C, D, E, F and G, comprising 452 target structures/proteins with few outliers and rendered in D3 force directed graphs. Each node represents the target/protein and each edge represents a molecular interaction between the two nodes from different clusters. All molecular interactions are clustered using graph-embedded self-clustering algorithms based on the random-walk and neighbor likelihood score.

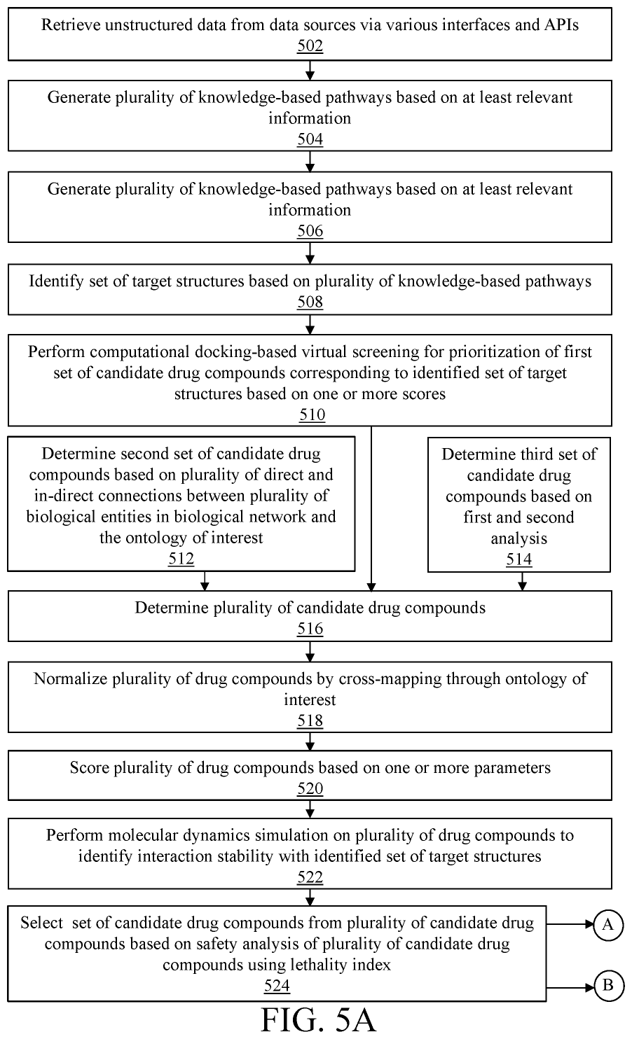

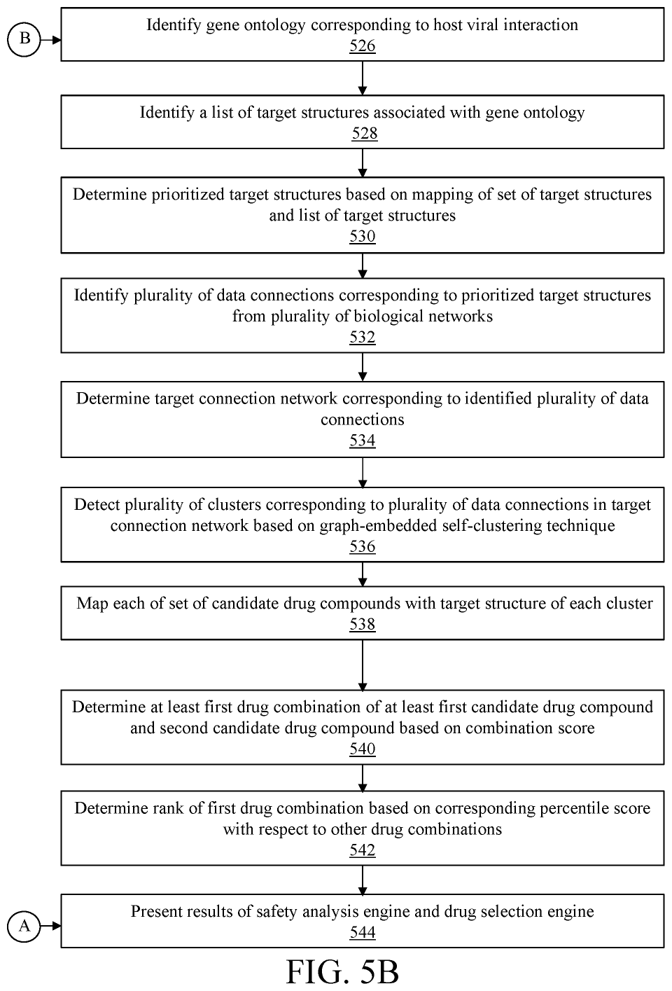

FIGS. 5A and 5B collectively depict flowcharts illustrating exemplary operations for selection of a set of candidate drug compounds, in accordance with an exemplary embodiment of the disclosure. Specifically, flowchart 500A depicts a method for selection of a set of candidate drug compounds, in accordance with an embodiment of the disclosure. Flowchart 500B depicts a method for selecting a combination of drug compounds, in accordance with another embodiment of the disclosure.

At step 502, unstructured data may be retrieved from the data sources 104. In accordance with an embodiment, the knowledge processing engine 107 may be configured to retrieve the unstructured data from the data sources 104 via the set of interfaces 102a.

Various examples of the unstructured data may include, but not limited to, text like email messages, service-center transcripts, PowerPoint presentations, survey responses, news, research papers, scientific posters, patent data, patient medical records, authors names, webpages, PDF files, journals, documents, metadata, social media forums, posts, tweets, blogs, images like pdf, graphs, photos, x-rays/MRIs, audio files, recorded voice, music, video, machine data, log files, and sensor data.

At step 504, structured information may be extracted from the unstructured data based on one or more AI and NLP techniques. In accordance with an embodiment, the knowledge processing engines 107 may be configured to extract the structured information from the unstructured data based on one or more AI and NLP techniques. The structured information, thus generated, may include, but not limited to, a number of principal investigators, intervention used in clinical trials, expressions, biological functions, mutations and mechanism of actions retrieved from the relevant publications and the clinical trial registries associated with a medical condition of a host entity.

At step 506, a plurality of knowledge-based pathways may be generated based on at least the relevant information. In accordance with an embodiment, the pathway generation engine 110 may be configured to generate knowledge-based pathways based on at least the relevant information. In accordance with an embodiment, the relevant information may be extracted by the knowledge processing engines 107 from the structured information based on an ontology of interest. In an exemplary embodiment as described herein, the ontology of interest may correspond to life science ontology. Thus, the relevant information may correspond to a subset of the structured data, such as the number of principal investigators, intervention used in clinical trials, expressions, biological functions, mutations and mechanism of actions retrieved from the relevant publications and the clinical trial registries associated with COVID-19, that correspond to the life science ontology.

In accordance with the exemplary embodiment, the pathway generation engine 110 may be configured to generate a knowledge-based graphical network based on information of host factors co-opted during individual stages of infection replication. The knowledge-based graphical network may include a plurality of knowledge-based pathways, such as the first knowledge-based pathway 202a, the second knowledge-based pathway 202b, the third knowledge-based pathway 202c, and the fourth knowledge-based pathway 202d. The first knowledge-based pathway 202a may correspond to virus replication and host gene expression shut-off, the second knowledge-based pathway 202b may correspond to Endoplasmic Reticulum (ER) stress, the third knowledge-based pathway 202c may correspond to apoptosis and autophagy, and the fourth knowledge-based pathway 202d may correspond to innate immune system, as described in detail in FIG. 2. In accordance with the exemplary embodiment, the concepts corresponding to the plurality of knowledge-based pathways (as discussed above) are described hereunder. However, it may be noted that the below descriptions are merely for exemplary purposes (corresponding to COVID-19 infection) and should not be construed to limit the scope of the disclosure.

### Virus Replication and Host Gene Expression Shut-Off

Cell entry is an essential component of cross-species transmission, especially for the beta-coronaviruses. All coronaviruses encode a surface glycoprotein, spike (S) protein which binds to the host-cell receptor and mediates endocytosis of the coronaviruses into the host cell. Recently, the novel COVID-19 has been reported to use the SARS-coronavirus receptor ACE2 and the cellular protease TMPRSS2 for entry into target cells. Binding of the S protein to the receptor, triggers a conformational change in the S protein which leads in membrane fusion for viral entry, thereby delivering the nucleocapsid into the cytoplasm using the endosomal pathway and/or the cell surface non-endosomal pathway. The low pH and the pH-dependent endosomal cysteine protease cathepsin L may play an important role in endosomal viral entry by fusion of viral envelope to the cellular membrane. On the other hand, the type II transmembrane protease TMPRSS2 activates the spike (S) protein for cell surface non-endosomal virus entry at the plasma membrane.

Once into the host cell, the viral genome is translated into two large polyproteins, pp1a and pp1ab, which are auto proteolytically cleaved by virus-encoded proteases, the papain-like protease (PLpro) and the 3C-like protease (3CLpro) to produce nonstructural proteins (nsps) with diverse functions. At the same time, polymerase, which produces a nested set of sub genomic RNA (sgRNA) species by discontinuous transcription, is finally translated into relevant structural and accessory viral proteins. These proteins are subsequently assembled into virions in the endoplasmic reticulum (ER) and Golgi, which are budded into the ER-Golgi intermediate compartment and then transported inside smooth-wall vesicles and released out of the cell via the secretory pathway.

In addition to its replication, the viruses also suppress the host gene expression, a process that is referred to as host shutoff. Accordingly, the viruses may limit the production of antiviral proteins and increase production capacity for viral proteins.

In SARS-CoV, nonstructural protein 1 (nsp1) is the key factor in virus-induced down-regulation of host gene expression. Specific interaction of nsp1 with the 5′ untranslated region (UTR) of SARS-CoV mRNA protects viral mRNAs from nsp1-mediated translational shutoff in SARS-CoV-infected cells. Moreover, nsp1 significantly altered the nuclear pore complex by disrupting Nup93 localization around the nuclear envelope without triggering proteolytic degradation of the protein while other nucleoporins and the nuclear lamina remain unperturbed. Consistent with its role in host shutoff, nsp1 alters the nuclear-cytoplasmic distribution of a RNA binding protein, nucleolin.

### ER Stress

ER is the major site for synthesis and folding of secreted or membrane proteins. SARS-CoV S glycoprotein, relies heavily on the ER protein chaperones and modifying enzymes for its folding and maturation. When the ER capacity for folding and processing proteins is accumulated, unfolded or misfolded proteins rapidly accumulate in the lumen leading to ER stress. To adjust the biosynthetic burden and capacity of the ER for maintaining cellular homeostasis, a complex signaling pathway known as unfolded protein response (UPR) is activated. However, under prolonged ER stress, UPR can also induce apoptotic cell death. The UPR pathway is mediated by three distinct signaling tracks initiated by the transmembrane sensors, known as activating transcription factor 6 (ATF6), inositol-requiring enzyme 1 (IRE1), and protein kinase RNA-activated (PKR)-like ER protein kinase (PERK). Activated ATF6α is transported to the Golgi apparatus and its cytosolic domain is cleaved by SIP and S2P proteases, which triggers the transcription of the ER protein chaperones (GRP78, GRP94). On the other hand, activated IRE1α dimerization and phosphorylation induces XBP1 mRNA splicing to generate active XBP1s, which increases the expression of UPR functional genes. PERK phosphorylates the downstream translation initiation factor eIF2α, leading to the attenuation of overall protein translation and the activation of ATF4, which activates the expression of CHOP. Under ER stress conditions, the XBP1, ATF4, and ATF6α transcription factors are translocated to the nucleus where they actuate the expression of target genes. Activation of the three branches of UPR modulates a wide variety of cellular processes such as; Apoptosis, Autophagy, and Innate Immune Response.

### Apoptosis and Autophagy

Induction of immune cells apoptosis in HCoV diseases, such as SARS, contribute to the suppression of host immune response. Both intrinsic (mitochondrial) and extrinsic (death receptor) pathways are activated upon HCoV infection. Persistence of ER stress may lead to an increase in expression of GADD153 resulting in mitochondrial dependent apoptosis by altering the Bax/Bcl-2 ratio and cytochrome c release from mitochondria. Cytosolic cytochrome c binds to APAF-1, which forms a complex with procaspase-9 leading to activation of caspase-9 and cell death. In the death receptor pathway, the binding of a ligand to its death receptor recruits an adaptor protein that in turn activates procaspase-8. FasL binds to Fas that activates FADD. FADD activates caspase-8. Caspases-8 and -9 in turn activate caspase-3. Caspase-3 plays a crucial role in the promotion of apoptotic cell death.

Autophagy is cellular response to starvation, whereby cells eliminate damaged or diseased components in order to regenerate and build new healthier cells. Thus, viruses are usually identified and disposed of in this way. Under stimulatory conditions, MTOR is inactivated, the ULK complex becomes hypophosphorylated and relocates to the site of formation of the autophagosome, the phagophore.

### Innate Immune System

The effective innate immune response signaling cascade starts with the recognition of the invasion of the virus by pattern recognition receptors (PRRs). For RNA virus such as COVID-19, viral genomic RNA or the intermediates during viral replication including dsRNA, are recognized by either the endosomal RNA receptors, TLR3/7 and the cytosolic RNA sensor, RIG-I/MDA5. TLR3 and TLR7 upon recognition of the endosomal dsRNA and ssRNA, respectively signals through the myeloid differentiation primary response gene 88 (MyD88) pathway.

This recognition triggers induction of the following four transcription factors: nuclear factor kappa-light-chain-enhancer of activated B cells (NF-κB), activator protein 1 (AP-1), and interferon regulatory factors 3 and 7 (IRF3 and IRF7). In the nuclei, these transcription factors are involved in the regulation of IFN expression, while NF-κB and AP-1 are involved in the induction of other pro-inflammatory cytokines (TNF-alpha, IL-1, IL-6). These initial responses comprise the first line defense against viral infection at the entry site. Type I IFN via IFNAR, in turn, activates the JAK-STAT pathway, where JAK1 and TYK2 kinases phosphorylate STAT1 and STAT2. STAT1/2 form a complex with IRF9, and together they move into the nucleus to initiate the transcription of IFN-stimulated genes (ISGs) under the control of IFN-stimulated response elements (ISRE) containing promoters. A successful mounting of this type I IFN response may suppress viral replication and dissemination at an early stage.

At step 508, a set of target structures may be identified based on the plurality of knowledge-based pathways. In accordance with an embodiment, the pathway generation engine 110 may be configured to identify the set of target structures based on the plurality of knowledge-based pathways. In accordance with an embodiment, the pathway generation engine 110 may be further configured to identify a set of target structures based on the plurality of knowledge-based pathways, such as the first knowledge-based pathway 202a, the second knowledge-based pathway 202b, the third knowledge-based pathway 202c, and the fourth knowledge-based pathway 202d, as described in FIG. 2.

In accordance with the exemplary embodiment, the identified set of target structures may correspond to the host protein and the virus protein in case of a specific medical condition, such as viral infection. Examples of the set of target structures may include, for example, angiotensin-converting enzyme-2 (ACE2) 204, Transmembrane Protease Serine-2 (TMPRSS2) 206, Eukaryotic Initiation Factor 2 alpha (eIF2α) 208, Inositol-requiring enzyme-1 (IRE1) 210, Activating Transcription Factor-6 (ATF6) 212, interleukin-1 receptor-associated kinase 4 (IRAK4) 214, RNA-dependent RNA polymerase (RdRp) 216, and papain-like protease (PLpro) 218 and the 3C-like protease (3CLpro) 220, as illustrated in FIG. 2. In accordance with the exemplary embodiment, as the set of target structures play an important role in the viral entry, host-interaction, replication, ER stress and innate immune system, as described above, therefore the set of target structures may be considered as potential therapeutic target structures for the identification of therapeutic interventions against COVID-19 infection.

At step 510, a computational docking-based virtual screening may be performed for prioritization of a first set of candidate drug compounds corresponding to the identified set of target structures based on one or more scores. In accordance with an embodiment, the screening engine 114 may be configured to perform the computational docking-based virtual screening for the prioritization of the first set of candidate drug compounds corresponding to the identified set of target structures based on the one or more scores.

In an example, the computational docking-based virtual screening approach was performed on approximately 1600 drugs, potential diverse and active inhibitors identified for the set of target structures. In accordance with the exemplary embodiment, the concepts corresponding to the computational docking-based virtual screening approach are described hereunder. However, it may be noted that the below descriptions are merely for exemplary purposes (corresponding to COVID-19 infection) and should not be construed to limit the scope of the disclosure.

### Receptors and Ligand Preparation

As indicated in Table 1 below, eight target structures may be selected from the pathway analysis of viral host interaction evident for COVID-19. The three-dimensional (3D) structures of all the target structures except TMPRSS2 may be retrieved from Protein Data Bank (PDB). The PDB id RdRp and 3CLPro protein is the same, as both of them belong to the same family and pathway. Cases where multiple crystal entries have been identified for a given target structure, preference may be given to structure entry where (1) a drug-like molecule is co-crystallized and (2) resolution of structure entry is good. In order to perform virtual screening, the protein files may be prepared for AutoDockTools® by removing the cocrystal ligands. Water molecules from the structure, hydrogen atoms and partial charges (Gasteiger) may be added, and the coordinates of the 3D structures may be saved in pdbqt format for further molecular docking process. Grid of the proteins may be generated by using the cocrystal ligands as the reference. The 3D structure of top listed drugs for identified proteins may be downloaded from PubChem® and the structure may be minimized and converted to pdb format using Open babel®. UCSF Chimera® may be used for visualization of the docked poses.

TABLE 1
Details of target structures details selected for docking studies

| | | | | | Crucial | |
|---|---|---|---|---|---|---|
| Target | Target | Uniprot | PDB | Resolutions | residues | Potential |
| Name | Class | ID | ID | (Å) | (Active sites) | compounds |
| ACE-2 | Protease | Q9BYF1 | 1R4L | 3.0 | Arg273, His345, | 792 |
| | | | | | Pro346, Thr371, | |
| | | | | | Glu375, Glu402, | |
| | | | | | Tyr515 | |
| TMPRSS2 | Protease | O15393 | — | — | — | |
| eIF2α | Nuclear | P05198 | 6O81 | 3.21 | E:Ser178, | 399 |
| | Protein | | | | F:Ser178 | |
| IRE-1 | Kinase | O75460 | 4U6R | 2.5 | Glu651, Cys645, | 399 |
| | | | | | Asp711, Phe712 | |
| IRAK4 | Kinase | Q9NWZ3 | 5UIU | 2.02 | Val263, Met265, | 404 |
| | | | | | Ala315, Ser328 | |
| RdRp | Protease | P0C6X7 | 6JJJ | 2.65 | Gly141 | 399 |
| 3CLPro | Protease | P0C6X7 | 6JJJ | 2.65 | Gly141 | 399 |
| PLpro | Protease | K4LC41 | 5YNM | 1.68 | Asn43, Gly81, | |
| | | | | | Gly71, Gly73, | |
| | | | | | Asp99, Leu100, | |
| | | | | | Cys115, Asp130 | |

### Protein Preparation, Selection of Binding Site, Ligand Preparation and Running the Virtual Screening Campaign

Autodock vina 1.1.2 @ may be used to perform the docking based virtual screening of approximately 1600 potential candidate drug compounds against the X-Ray structure of the selected proteins listed in Table 1. As the crystal structure of TMPRSS2 protein is not available in the PDB database so screening may be not performed for such protein. For preparation of protein receptors and screening chemical libraries, AutoDockTools® may be used. Target structures may be loaded individually and Hydrogens may be added using the tool. Gasteiger charges may be added, unwanted crystal adducts may be deleted and pdbqt file may be saved. The bound crystal ligand of individual target structure may be used as a reference for the selection of binding sites. AutoDockTools® may be also used for the energy minimization of compounds and for converting all molecules to AutoDock Ligand format (PDBQT). Standard grids may be generated for all the selected proteins based on their critical binding residues as mentioned in Table 1, such as for ACE-2 protein using Arg273, His345, Pro346, Thr371, Glu375, Glu402, Tyr515 amino acids and its cocrystal inhibitor. Similarly, grids for IRAK4 may be generated by using the Val263, Met265, Ala315, Ser328 amino acids and a potent, selective cocrystal clinical candidate, having the IC50 value of 0.2 nM for IRAK4. Calculations may be performed in a high-performance computing environment using proprietary scripts.

In accordance with an exemplary embodiment, the screening engine 114 may be configured to perform the computational docking-based virtual screening on the selected set of target structures, i.e. 8 structures, and prioritize the first set of candidate drug compounds, i.e. 14 drug compounds, as highly potential candidates for COVID-19. The prioritization of 14 compounds may be based on one or more scores. A first score of the one or more scores may be a quantitative docking score that corresponds to performance of each candidate drug compound for each target structure. A second score of the one or more scores may be an affinity score that corresponds to an overall strength of binding affinity of each candidate drug compound based on a spatial arrangement of docking pose and presence of hydrogen bond interactions with each target structure.

Accordingly, against the target structure IRAK4, the second score of each of the 14 drug compounds is the highest. Against the target structure eIF2α, the second score of 7 out of 14 drug compounds is the highest. Similarly, against the target structure IRE1, the second score of 5 out of 14 drug compounds is the highest.

In accordance with the exemplary embodiment, out of the 14 drug compounds, Maraviroc, Carfilzomib, Darunavir, Telmisartan and Medroxyprogesterone may be prioritized. The 5 drugs efficiently bind in the active site pocket of the target structures and illustrate good overlapping with the cocrystal ligands/drugs. Hydrogen bond (H-bond) interacting distances range from 1.8 to 3.8 Å and the H-bond numbers are from 2 to 6 for the 8 target structures.

In accordance with the exemplary embodiment, Table 2 below provides a prioritized first set of candidate drug compounds from the computational docking-based virtual screening from the existing drug molecules with RdRp, IRE-1, IRAK4, ACE-2, elF2α and PLpro molecules with corresponding docking score, average percentile of network score, and safety score. Table 2 below is sorted based on the final cumulative score obtained from the molecular docking score, the safety score, and the network score.

TABLE 2
Prioritized first set of candidate drug compounds

| | | | | | | | Avg | Avg | | | |
|---|---|---|---|---|---|---|---|---|---|---|---|
| | | | | | | | percentile | percentile | | | |
| | | | | | | | (Affinity | (Network | Safety | Final | |
| Drug Name | RdRp | IRE-1 | IRAK:4 | ACE-2 | eIF2α | PLpro | score) | score) | score | Score | Originator |
| Maraviroe | 100 | 100 | 100.00 | 82.05 | 100.00 | 95.48 | 95.51 | 57.7001 | 83.77 | 78.99 | Pfizer |
| Hydrocortisone | 73.33 | 70.74 | 70.85 | 54.36 | 67.01 | 74.58 | 68.48 | 80.872 | 83.11 | 77.49 | Generic, |
| | | | | | | | | | | | Edward |
| | | | | | | | | | | | Kendall |
| Medroxyprogesterone | 79.33 | 73.94 | 80.90 | 56.41 | 74.11 | 68.93 | 72.27 | 66.572 | 86.49 | 75.11 | Pfizer |
| | | | | | | | | | | | (Generic) |
| Simvastatin | 62.00 | 67.02 | 63.32 | 51.28 | 57.87 | 58.19 | 59.95 | 79.666 | 84.51 | 74.71 | Merck and |
| | | | | | | | | | | | Schering- |
| | | | | | | | | | | | Plough |
| Telmisartan | 81.33 | 79.79 | 78.39 | 77.44 | 74.62 | 76.27 | 77.97 | 60.645 | 83.08 | 73.90 | Boehringer |
| | | | | | | | | | | | Ingelheim |
| Isotretinoin | 60.67 | 58.51 | 57.79 | 37.95 | 55.84 | 58.76 | 54.92 | 79.347 | 85.97 | 73.41 | Generics |
| | | | | | | | | | | | (Roche |
| | | | | | | | | | | | Holding |
| | | | | | | | | | | | AG) |
| Losartan | 74.67 | 70.21 | 73.87 | 63.08 | 64.47 | 67.80 | 69.01 | 64.833 | 83.43 | 72.43 | Bristol- |
| | | | | | | | | | | | Myers |
| | | | | | | | | | | | Squibb |
| Baricitinib | 61.33 | 56.38 | 56.28 | 46.15 | 54.82 | 63.28 | 56.37 | 74.059 | 86.04 | 72.16 | Eli Lilly |
| | | | | | | | | | | | and |
| | | | | | | | | | | | Company |
| Trans-resveratrol | 48.67 | 44.68 | 48.74 | 48.21 | 44.67 | 48.02 | 47.16 | 73.314 | 93.48 | 71.32 | Generic |
| Plerixafor | 78.67 | 68.09 | 74.37 | 75.38 | 65.99 | 68.93 | 71.90 | 55.393 | 79.72 | 69.01 | Sanofi- |
| | | | | | | | | | | | Genzyme |
| Tofacitinib | 53.33 | 47.87 | 47.74 | 44.62 | 47.72 | 48.59 | 48.31 | 74.059 | 84.62 | 69.00 | Pfizer |
| Darunavir | 90.00 | 84.57 | 94.47 | 57.95 | 80.71 | 84.75 | 82.07 | 29.205 | 82.11 | 64.46 | Johnson & |
| | | | | | | | | | | | Johnson |
| Trametinib | 79.33 | 68.09 | 78.89 | 73.85 | 69.54 | 74.01 | 73.95 | 28.921 | 83.23 | 62.03 | GSK |
| Carfilzomib | 90.00 | 90.43 | 88.44 | 80.51 | 83.76 | 75.71 | 84.81 | 16.784 | 81.71 | 61.10 | Onyx |
| | | | | | | | | | | | Phar- |
| | | | | | | | | | | | maceuticals |

At step 512, a second set of candidate drug compounds may be determined based on plurality of direct and in-direct connections between a plurality of biological entities in a biological network and the ontology of interest. In accordance with an embodiment, the search engine 112, in conjunction with the knowledge processing engines 107, may be configured to determine the second set of candidate drug compounds based on the plurality of direct and in-direct connections between the plurality of biological entities in the biological network and the ontology of interest.

In accordance with the exemplary embodiment, the knowledge processing engines 107 may be configured to determine the second set of candidate drug compounds. The second set of candidate drug compounds may be non-obvious potential candidate drug compounds for the selected 8 target structures. The knowledge processing engines 107 may leverage the search engine 112, i.e. Ontosight Explore®, which is an ontology-based biological network of protein, pathways, drugs and diseases. For instance, in order to identify potential candidate drug compounds, the interactions flow is—protein interacts with pathways, pathways interact with disease and disease interacts with drugs. Ontosight Explore® works on the concepts that if entity 1 is connected to entity 2 and entity 2 is connected to entity 3 and 4, entity 1 has indirect connections with entity 4 which may be scored based on a number of parameters, such as druggability, druglikeness and publicly available evidence from literature, patents, grants, thesis, news and press evidence. Such scoring, as illustrated as labels on each molecular interaction in FIG. 3, may prioritize most potential candidate drug compounds, i.e. the second set of candidate drug compounds, for the set of 8 targets.

In an exemplary embodiment, the search engine 112, i.e. Ontosight Explore®, may yield 1,606 number of therapeutic interventions from the set of target structures. For all the selected seven protein target structures, 201 number of associated biological pathways and 1,606 number of potential candidate drug compounds may be identified. Identified drug molecules may be ranked based on the association score and grouped based on the identified therapeutic targets for COVID-19 which includes ACE2 inhibitors (352), TMPRSS2 inhibitors (397), IRE-1 inhibitors (344), ATF6 inhibitors (395), eIF2α inhibitors (390) and IRAK4 inhibitors (383) RdRp inhibitors (364). For example, the top drug compound may be identified to be ‘Maraviroc/which is associated with 150 associated pathways and having 760 interactions with other biological molecules.

At step 514, a third set of candidate drug compounds may be determined based on analysis of gene and protein expression profile of the identified set of target structures. In accordance with an embodiment, the expression analysis engine 116 may be configured to determine the third set of candidate drug compounds based on the first analysis of gene and protein expression profile of the identified set of target structures, and a second analysis of expression profiles of the third set of candidate drug compounds and corresponding pharmacokinetics effect.

In an exemplary embodiment, the expression analysis engine 116 may perform the analysis based on literature mining. The expression analysis engine 116 may perform ontology-based search in the unstructured data for specific drug modulation(s) in the identified set of target structures, for example drug ‘x’ up-regulate or downregulate the ‘Y’ target structures in a Covid-19 patient. In an exemplary embodiment, the expression analysis engine 116 may perform the analysis based on extraction of similar disease sample, such as SARS CoV, MERS, and the like, for a target disease, such as Covid-19, and identify treated drug compound(s) and corresponding responder genes/proteins.

At step 516, the plurality of candidate drug compounds may be determined. In accordance with an embodiment, the ANS engine 118 may be configured to determine the plurality of candidate drug compounds. The plurality of candidate drug compounds may be determined based on the first, second and third set of candidate drug compounds from the screening engine 114, the search engine 112, and the expression analysis engine 116, respectively.

At step 518, the plurality of candidate drug compounds may be normalized by cross-mapping through the ontology of interest from the set of ontologies 108. In accordance with an embodiment, the ANS engine 118 may be configured to normalize the plurality of candidate drug compounds by cross-mapping through the ontology of interest from the set of ontologies 108.

At step 520, the plurality of candidate drug compounds may be scored based on one or more parameters. In accordance with an embodiment, the ANS engine 118 may be configured to score the plurality of candidate drug compounds based on the one or more parameters. Examples of the one or more parameters may include, but not limited to, clinical trials for a specific disease, such a Covid-19 (Exists—0/No Exists—1), a safety score of a drug compound (Tolerable Adverse events—1, Severe adverse events—0), expression profiles (Drug respond to the identified set of target structures?), approved drug compound (Other indication) or novel drug compound s (Approved—1, Novel—0, Clinical drug—1), patent evidence for drug repurposing (No—1, Yes—0), literature evidences for any COVID-19 similar virus (Yes—1, No—0), and cumulative scores of above mentioned evaluation parameters.

At step 522, molecular dynamics simulation may be performed on the plurality of candidate drug compounds to identify their interaction stability with identified set of target structures. In accordance with an embodiment, the molecular stability analysis engine 120 may be configured to perform the molecular dynamics simulation on the plurality of candidate drug compounds to identify their interaction stability with identified set of target structures. The most stable proteins and drug compound combinations may be selected based on protein-ligand complex RMSD values.

At step 524, a set of candidate drug compounds may be selected from the plurality of candidate drug compounds based on safety analysis of the plurality of candidate drug compounds using a lethality index. In accordance with an embodiment, safety analysis engine 122 may be configured to select the set of candidate drug compounds from the plurality of candidate drug compounds based on the safety analysis of the plurality of candidate drug compounds using the lethality index.

In accordance with an embodiment, the safety analysis engine 122 may perform the safety analysis using an adverse event analysis protocol, such as lethality index. In accordance with an embodiment, the lethality index is a scatter plot with safety coordinates which efficiently positions adverse events on ‘X’ and ‘Y’ axis, such as ULI versus UFI respectively. The ULI and UFI may be calculated based on publicly available adverse events, severity, frequency and outcome within a specific time frame.

In accordance with an embodiment, the control may proceed to step 524 in flowchart 500B of FIG. 5B to display the results of the safety analysis engine 122.

In accordance with another embodiment, the control may proceed to step 526 in flowchart 500B of FIG. 5B to determine one or more drug combinations.

With reference to flowchart 500B, at step 526, a gene ontology corresponding to a host viral interaction may be identified. In accordance with an embodiment, the search engine 112 may be configured to identify the gene ontology corresponding to the host viral interaction.

In an exemplary embodiment, in order to identify host viral interaction proteins, all the Gene Ontologies from GO database, such as mitigation of host defence by virus and modulation by virus of host process, and the like, may be collected. In accordance with the exemplary embodiments, various biological processes of virus, such as endocytosis involved in viral entry into host cell (GO:0075509), Suppression by virus of host adaptive immune response (GO:0039504), Modulation by virus of host protein ubiquitination (GO:0039648), Positive regulation by symbiont of host receptor-mediated endocytosis (GO:0044078) and Ubiquitin-dependent protein catabolic process (GO:0006511), may be considered.

At step 528, a list of target structures associated with the gene ontology may be identified. In accordance with an embodiment, the search engine 112 may be configured to identify the list of target structures associated with the gene ontology.

At step 530, prioritized target structures may be determined based on mapping of the set of target structures and list of target structures. In accordance with an embodiment, the search engine 112 may be configured to determine the prioritized target structures based on mapping of the set of target structures and the list of target structures. In the exemplary embodiment, the target structures may be prioritized by mapping the set of target structures and the list of target structures. More weightage may be provided to target structures that are present in both the set of target structures and the list of target structures. Further only such proteins may be considered that are associated with ‘host viral interaction’ mechanisms that may be targeted. Proteins involved in more than two host viral interactions may be provided more weightage.

At step 532, a plurality of data connections may be identified corresponding to the prioritized target structures from the plurality of biological networks. In accordance with an embodiment, the network analysis engine 124 may be configured to identify the plurality of data connections corresponding to the prioritized target structures from the plurality of biological networks.

In accordance with the exemplary embodiment, 1.2 lacs of data connections may be identified from the plurality of biological networks against 452 target structures.

At step 534, a target connection network corresponding to the identified plurality of data connection may be determined. In accordance with an embodiment, the network analysis engine 124 may be configured to determine the target connection network corresponding to the identified plurality of data connections.

At step 536, a plurality of clusters corresponding to the plurality of data connections may be detected in the target connection network based on a graph-embedded self-clustering technique. In accordance with an embodiment, the clustering engine 126 may be configured to detect the plurality of clusters, such as the clusters illustrated in FIGS. 4A and 4B, corresponding to the plurality of data connections in the target connection network based on the graph-embedded self-clustering technique.

In accordance with the exemplary embodiment, the clustering engine 126 may be configured to detect 6 major clusters for 452 target structures with few outliers, as illustrated in FIG. 4B.

At step 538, each of the set of candidate drug compounds may be mapped with a target structure of each cluster. In accordance with an embodiment, the clustering engine 126 may be configured to map each of the set of candidate drug compounds with the target structure of each cluster.

In accordance with the exemplary embodiment, each target structure of a cluster may be mapped with approved drug compounds followed by classification of the drug compounds into eight groups based on the target clusters. For example, 12 drug compounds may be mapped with the proposed 14 drug compounds and may be used for further combination prioritization. Such 12 drugs lie in five different clusters while some drugs may be associated with more than one cluster group targets, as indicated in Table 1. Each cluster corresponds to a group of drug compounds which may be combined with another group.

At step 540, at least a first drug combination of at least a first candidate drug compound and a second candidate drug compound may be determined based on a combination score. In accordance with an embodiment, the drug selection engine may be configured to determine at least the first drug combination of at least the first candidate drug compound and the second candidate drug compound based on the combination score. In accordance with the exemplary embodiment, the first drug combination may be determined based on multiple permutation and combinations of inter cluster drug compounds.

With reference to Table 3 below, there are shown identified repurposed drugs.

TABLE 3
Eight different clusters identified using target clustering followed by drug mapping.

| Cluster | | | | |
|---|---|---|---|---|
| name | Drug name | | | |
| Drug | Maraviroc | Loratadine | Vismodegib | Atectimib |
| associated | | | | |
| with | Pentostatin | Amifostine | Carmustine | Nitroglycerin |
| A | | | | |
| cluster | | | | |
| Drug | Candesartan | Losartan | Abiraterone | Teriflunomide |
| assoeiated | | | | |
| with | | | | |
| B | | | | |
| cluster | | | | |
| Drug | Warfarin | Cyclophosphamide | Ifosfamide | |
| associated | | | | |
| with | | | | |
| C | | | | |
| cluster | | | | |
| Drug | Rimonabant | Ciofazimine | Cerivastatin | Carfilzomib |
| associated | Omeprazole | Diltiazem | Etoposide | Metolazone |
| with | Aprepitant | Ciprofloxacin | Mitoxantrone | Lansoprazole |
| D | | | | |
| cluster | | | | |
| Drug | Quinine | Rivaroxaban | Torasemide | Tolazamide |
| associated | | | | |
| with | | | | |
| E | | | | |
| cluster | | | | |
| Drug | Rimonabant | Clofazimine | Cerivastatin | Carfilzomib |
| associated | Aprepitant | Ciprofloxacin | Omeprazole | Diltiazem |
| with | Idarubicin | Chlorothiazide | Mitoxantrone | Lansoprazole |
| F | | | | |
| cluster | | | | |
| Drug | Obatoclax | Naltrexone | Capsaicin | Clarithromycin |
| asociated | Belinostat | Ibrutinib | Anakinra | Cilostazol |
| with | Vemurafenib | | | |
| G | | | | |
| cluster | | | | |
| Drug | Epothilone B | Nialamide | 4,7,10,13,1 | Triclosan |
| Cluster | | | 6,19- | |
| 8 | | | docosahexaenoic | |
| (A & G | | | acid | |
| clusters) | Perhexiline | Medroxyprogesterone | Liothyronine | Doxofylline |
| | Aripiprazole | Apraclonidine | Binimetinib | Prazosin |
| | Telmisartan | Dronedarone | RPL | Lovastatin |
| | Carvedilol | Dexamethasone | Pitavastatin | Trametinib |
| | Fluvastatin | Topiramate | Abemaciclib | Pravastatin |
| | Baricitinib | Methylprednisolone | Ketorolac | Tolvaptan |
| | Ursodeoxycholic | Sertraline | Simvastatin | Zolmitriptan |
| | acid | | | |
| | Lapatinib | Ropinirole | Ranolazine | Bexarotene |
| | Flecainide | Fentanyl | Sorafenib | Tretinoin |
| | Sitaxentan | Axitinib | Cefazolin | Vatalanib |
| | Hydrochlorothiazide | Hydroxychloroquine | Bosentan | Rosiglitazone |
| | Procainamide | Enalaprilat | Captopril | Terbutaline |
| | Pomalidomide | Tegaserod | Treprostinil | Fingolimod |
| | Epoprostenol | Veliparib | Pramipexole | Ranitidine |
| Cluster | | | | |
| name | Drug name | | | |
| Drug | Chlorhexidine | Idelalisib | Hydroxyzine | Prasugrel |
| associated | gluconate | | | |
| with | | | | |
| A | | | | |
| cluster | | | | |
| Drug | Midazolum | Plerixafor | Voglibose | |
| assoeiated | | | | |
| with | | | | |
| B | | | | |
| cluster | | | | |
| Drug | | | | |
| associated | | | | |
| with | | | | |
| C | | | | |
| cluster | | | | |
| Drug | Moxifloxacin | Chloramphenicol | Flutemetamol F 18 | Levofloxacin |
| associated | Methyldopa | Daunorubicin | Deferoxamine | Idarubicin |
| with | Chlorothiazide | | | |
| D | | | | |
| cluster | | | | |
| Drug | Thiamine | Azacitidine | Decitabine | |
| associated | | | | |
| with | | | | |
| E | | | | |
| cluster | | | | |
| Drug | Moxifloxacin | Chloramphenicol | Flutemetamol F 18 | Levofloxacin |
| associated | Etoposide | Metolazone | Methyldopa | Daunorubicin |
| with | Deferoxamine | | | |
| F | | | | |
| cluster | | | | |
| Drug | Ergocalciferol | Alpelisib | Cholecalciferol | Calcitriol |
| asociated | Rivastigmine | Levamisole | Panobinostat | Enoximone |
| with | | | | |
| G | | | | |
| cluster | | | | |
| Drug | Montelukast | Fostamatinib | Fluticasone | Desvenlafaxine |
| Cluster | Bromocriptine | Lisuride | Doxarosin | Hexachlorophene |
| 8 | Ketoconazole | Flavopiridol | Budesomide | Sapropterin |
| (A & G | Imatinib | Minocyclin | Terazosin | Cabozantinib |
| clusters) | Sulindac | Celecoxib | Morphine | Midostaurin |
| | Vandetanib | Ipratropiumbromide | Palbociclib | Fenofibrate |
| | Triamterene | Hydrocortisone | Isotretinoin | Disopyramide |
| | Epirubicin | Dofetilide | Nicardipine | Gliclazide |
| | Glimepiride | Verapamil | Perindopril | Bupropion |
| | Crizotinib | Propafenone | Levosimendan | Cannabidiol |
| | Lidocaine | Propranolol | Amiodarone | Dasatinib |
| | Trimethoprim | Lenvatinib | Metoclopramide | Misoprostol |
| | Azathioprine | Gemcitabine | Dobutamine | Amiloride |
| | Salbutamol | Sotalol | Lenalidomide | Disulfiram |
| | Adenosine | Glutathione | Pralatrexate | Romidepsin |
| | triphosphate | | | |

In order to determine a prioritized combination drug compound for identified repurposed drugs indicated in Table 3 above, a combination score may be determined using the docking score of individual drug compounds and target structure along with corresponding lethality score and safety score, indicated in Table 2 above. To calculate the combination score, the average safety score of all drug compounds in combination may be divided by average lethality score. Thereafter, average percentile docking score may be divided by that score as mathematically expressed as equation (2) above.

With reference to Table 4a, 4b, and 4c below, there are shown various drug combinations for drug compounds ‘Maraviroc’, ‘Carfilzomib’, and ‘Plerixafor’ as exemplar use cases. The combination scores are calculated based on docking scores, lethality scores and safety scores. Pharmacological action of both the drugs also mapped in the last two columns of each of Tables 4a, 4b, and 4c.

TABLE 4a
Drug compound combination table with one drug compound as ‘Maraviroc’.

| | | Cumulative | Pharmacological | Pharmacological action |
|---|---|---|---|---|
| Drug One | Drug Two | combination score | action (Drug One) | (Drug two) |
| Hydrocortisone | Maraviroc | 0.163 | Anti-Inflammatory Agents | HIV Fusion Inhibitors |
| | | | | CCR5 Receptor |
| | | | | Antagonists |
| Isotretinoin | Maraviroc | 0.134 | Dermatologic Agents | HIV Fusion Inhibitors |
| | | | Teratogens | CCR5 Receptor |
| | | | | Antagonists |
| Maraviroc | Carfilzomib | 0.188 | HIV Fusion Inhibitors | Antineoplastic Agents |
| | | | CCR5 Receptor Antagonists | ubiquitin-proteasome |
| | | | | Inhibitors |
| | Plerixafor | 0.187 | HIV Fusion Inhibitors | Anti-HIV Agents |
| | | | CCR5 Receptor Antagonists | |
| | Anakinra | 0.183 | HIV Fusion Inhibitors | Antirheumatic |
| | | | CCR5 Receptor Antagonists | Agents |
| | Warfarin | 0.154 | HIV Fusion Inhibitors | Anticoagulants |
| | | | CCR5 Receptor Antagonists | Rodenticides |
| Medroxy- | Maraviroc | 0.1472 | Contraceptives, Oral, Hormonal | HIV Fusion Inhibitors |
| progesterone | | | Contraceptives, Oral, Synthetic | CCR5 Receptor |
| | | | | Antagonists |
| Simvastatin | Maraviroc | 0.1472 | Anticholesteremic Agents | HIV Fusion Inhibitors |
| | | | Hypolipidemic Agents | CCR5 Receptor |
| | | | Hydroxymethylglutaryl-CoA | Antagonists |
| | | | Reductase Inhibitors | |
| Telmisartan | Maraviroc | 0.1730 | Antihypertensive Agents | HIV Fusion Inhibitors |
| | | | Angiotensin II Type 1 | CCR5 Receptor |
| | | | Receptor Blockers | Antagonists |
| Tofacitinib | Maraviroc | 0.135 | Protein Kinase Inhibitors | HIV Fusion Inhibitors |
| | | | | CCR5 Receptor |
| | | | | Antagonists |
| | Maraviroc | 0.168 | Antineoplastic Agents | HIV Fusion Inhibitors |
| | | | Protein Kinase Inhibitors | CCR5 Receptor |
| | | | | Antagonists |
| Losartan | Maraviroc | 0.162 | Antiarrhythmic Agents | HIV Fusion Inhibitors |
| | | | Antihypertensive Agents | CCR5 Receptor |
| | | | Angiotensin II Type 1 Receptor | Antagonists |
| | | | Blockers | |
| Baricitinib | Maraviroc | 0.1356 | Janus kinases JAK1 and JAK2 | HIV Fusion Inhibitors |
| | | | inhibitor | CCR5 Receptor |
| | | | | Antagonists |

TABLE 4b
Drug compound combination table with one drug compound as ‘Carfilzomib’.

| | | Cumulative | Pharmacological action | Pharmacological action |
|---|---|---|---|---|
| Drug One | Drug Two | combination score | (Drug One) | (Drug two) |
| Carfilzomib | Plerixafor | 0.187 | Antineoplastic Agents | Anti-HIV Agents |
| | | | ubiquitin-proteasome Inhibitors | |
| | Warfarin | 0.154 | Antineoplastic Agents | Anticoagulants |
| | | | ubiquitin-proteasome Inhibitors | Rodenticides |
| Hydrocortisone | Carfilzomib | 0.163 | Anti-Inflammatory Agents | Antineoplastic Agents |
| | | | | ubiquitin-proteasome |
| | | | | Inhibitors |
| Isotretinoin | Carfilzomib | 0.134 | Dermatologic Agents | Antineoplastic Agents |
| | | | Teratogens | ubiquitin-proteasome |
| | | | | Inhibitors |
| Maraviroc | Carfilzomib | 0.188 | HIV Fusion Inhibitors | Antineoplastic Agents |
| | | | CCR5 Receptor Antagonists | ubiquitin-proteasome |
| | | | | Inhibitors |
| Medroxy- | Carfilzomib | 0.1484 | Contraceptives, Oral, Hormonal | Antineoplastic Agents |
| progesterone | | | Contraceptives, Oral, Synthetic | ubiquitin-proteasome |
| | | | | Inhibitors |
| Simvastatin | Carfilzomib | 0.1470 | Anticholesteremic Agents | Antineoplastic Agents |
| | | | Hypolipidemic Agents | ubiquitin-proteasome |
| | | | Hydroxymethylglutaryl-CoA | Inhibitors |
| | | | Reductase Inhibitors | |
| Telmisartan | Carfilzomib | 0.173 | Antihypertensive Agents | Antineoplastic Agents |
| | | | Angiotensin II Type 1 | ubiquitin-proteasome |
| | | | Receptor Blockers | Inhibitors |
| Tofacitinib | Carfilzomib | 0.134 | Protein Kinase Inhibitors | Antineoplastic Agents |
| | | | | ubiquitin-proteasome |
| | | | | Inhibitors |
| | Carfilzomib | 0.168 | Antineoplastic Agents | Antineoplastic Agents |
| | | | Protein Kinase Inhibitors | ubiquitin-proteasome |
| | | | | Inhibitors |
| Losartan | Carfilzomib | 0.162 | Antiarrhythmic Agents | Antineoplastic Agents |
| | | | Antihypertensive Agents | ubiquitin-proteasome |
| | | | Angiotensin II Type 1 | Inhibitors |
| | | | Receptor Blockers | |
| Baricitinib | Carfilzomib | 0.135 | Janus kinases JAK1 and JAK2 | Antineopiastic Agents |
| | | | inhibitor | ubiquitin-proteasome |
| | | | | Inhibitors |

TABLE 4c
Drug compound combination table with one drug compound as ‘Plerixafor’.

| | Drug | Cumulative | Pharmacological action | Pharmacological action |
|---|---|---|---|---|
| Drug One | Two | combination score | (Drug One) | (Drug two) |
| Carfilzomib | Plerixafor | 0.187 | Antineoplastic Agents | Anti-HIV Agents |
| | | | ubiquitin-proteasome Inhibitors | |
| Hydrocortisone | Plerixafor | 0.160 | Anti-Inflammatory Agents | Anti-HIV Agents |
| Isotretinoin | Plerixafor | 0.131 | Dermatologic Agents | Anti-HIV Agents |
| | | | Teratogens | |
| Maraviroc | Plerixafor | 0.187 | HIV Fusion Inhibitors | Anti-HIV Agents |
| | | | CCR5 Receptor Antagonists | |
| Medroxyprogesterone | Plerixafor | 0.1465 | Caceptives, Oral, Hormonal | Anti-HIV Agents |
| | | | Contraceptives, Oral, Synthetic | |
| Simvastatin | Plerixafor | 0.1435 | Anticholesteremic Agents | Anti-HIV Agents |
| | | | Hypolipidemic Agents | |
| | | | Hydroxymethylglutaryl-CoA | |
| | | | Reductase Inhibitors | |
| Telmisartan | Plerixafor | 0.171 | Antihypertensive Agents | Anti-HIV Agents |
| | | | Angiotensin II Type 1 Receptor | |
| | | | Blockers | |
| Tofacitinib | Plerixafor | 0.130 | Protein Kinase Inhibitors | Anti-HIV Agents |
| | Plerixafor | 0.165 | Antineoplastic Agents | Anti-HIV Agents |
| | | | Protein Kinase Inhibitors | |
| Baricitinib | Plerixafor | 0.132 | Janus kinases JAK1 and JAK2 | Anti-HIV Agents |
| | | | inhibitor | |

At step 542, a rank of the first drug combination may be determined based on a corresponding percentile score with respect to other drug combinations. In accordance with an embodiment, the drug selection engine may be configured to determine the rank of the first drug combination based on the corresponding percentile score with respect to other drug combinations.

At step 544, the results of the safety analysis engine 122 and the drug selection engine 128 may be presented. In accordance with an embodiment, the user interface 130 may be configured to present the results of the safety analysis engine 122 and the drug selection engine 128.

Thus, in accordance with an exemplary embodiment, not to be construed to be limiting the scope of the disclosure, the proposed method and system may identify 8 target structures (EIF2A, TMPRSS2, IRAK4, IRE1, RdRp, ACE2, 3CLPro, PLpro) to counteract COVID-19 infection. The 8 target structures are crucial for viral penetration and replication processes. Furthermore, 14 drug compounds (Maraviroc, Hydrocortisone, Medroxyprogesterone, Simvastatin, Telmisartan, Isotretinoin, Losartan, Baricitinib, Trans-resveratrol, Plerixafor, Tofacitinib, Darunavir, Trametinib, Carfilzomib) may be prioritized that may have optimum therapeutic potential for the identified 8 target structure. Safety analysis concluded that Plerixafor, Resveratrol and Maraviroc may be safe to be used in COVID-19 infection, as per the type of adverse events reported for them in the public domain. Further, proposed methods and systems may select combinational drug compounds for COVID-19 infection.

In accordance with an exemplary embodiment, as a first use case, Maraviroc may be prioritized as one of the best combinations based on combination score with 70 percentile being the cut-off. Maraviroc is a C-C chemokine receptor type 5 (CCR5) receptor antagonist which restricts the attachment of virus to the host CCR5 receptor. CCR5 shares the similar biological function of host cell entry along with Angiotensin-converting enzyme 2 (ACE2). Moreover, CCR5 and IRAK4 both play an important role in cytokine signaling in the immune system. The combination of Plerixafor with Maraviroc may inhibit the host-virus interaction and activate the immune response. Other proposed combinations of the drug compounds corresponding to the first use case may be (1) Maraviroc with Carfilzomib (2) Maraviroc with Hydroxychloroquine; and (3) Maraviroc with Losartan.

In accordance with another exemplary embodiment, as a second use case, Carfilzomib may be prioritized as one of the best combinations based on combination score with 70 percentile being the cut-off. Carfilzomib is a protease inhibitor, specifically inhibiting enzymatic activity of proteasome subunit beta (PSMB5). Carfilzomib not only impairs viral entry but also RNA synthesis and subsequent protein expression of different CoVs. PSMB5 shares the similar biological function of mRNA catabolism and MAPK cascade with IRE1. Thus combination of Maraviroc and Carfilzomib may not only inhibit the host-virus interaction, but also inhibit the replication of the virus inside the host cell and activate the immune response. The combination of Plerixafor with Carfilzomib may not only inhibit the host-virus interaction, but also inhibit the replication of the virus inside the host cell and activate the immune response. Other proposed combinations of the drug compounds corresponding to the second use case may be (1) Carfilzomib with Maraviroc and (2) Carfilzomib with Telmisartan.

In accordance with another exemplary embodiment, as a third use case, Plerixafor may be prioritized as one of the best combinations based on combination score with 70 percentile being the cut-off. Plerixafor, is a selective inhibitor of CXCR4 which plays an important role in the treatment of human immunodeficiency virus 45. CXCR4 shares the similar biological function of MAPK cascade and host entry along with IRE1 and TMPRSS2, respectively. The combination of Plerixafor with Maraviroc may inhibit the host-virus interaction and activate the immune response. Similarly, combination of Plerixafor with Carfilzomib may not only inhibit the host-virus interaction, but also inhibit the replication of the virus inside the host cell and activate the immune response. Other proposed combinations of the drug compounds corresponding to the third use case may be (1) Plerixafor with Trametinib (2) Plerixafor with Telmisartan (3) Plerixafor with Hydrocortisone and (4) Plerixafor with a combination of Trametinib, Telmisartan and/or Hydrocortisone.

Combination therapies may limit the viral infection by means of multiple mechanisms of actions like, viral attachment with a host receptor, restricting the viral replication inside the host, or restricting the nucleic acid synthesis. Combinational drug compounds may be precisely placed together considering corresponding particular mechanisms of actions, pathways, biological processes and safety profiles. Thus, by way of an example referring to the exemplary embodiment, combination of Plerixafor with Maraviroc might inhibit the host-virus interaction and activate the immune response. Similarly, combination of Plerixafor with Carfilzomib might not only inhibit the host-virus interaction, but also inhibit the replication of the virus inside the host cell and activate the immune response.

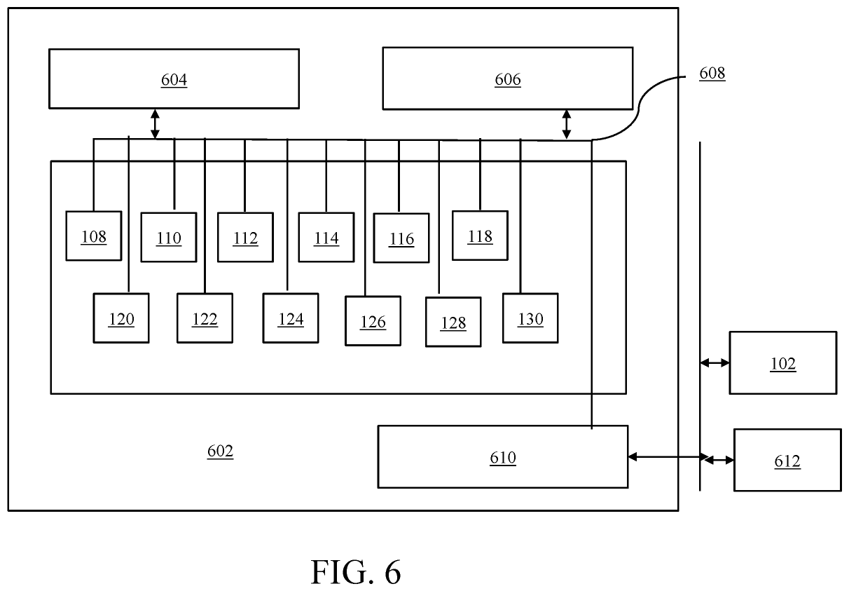

FIG. 6 is a conceptual diagram illustrating an example of a hardware implementation for a system employing a processing system for selection of a set of candidate drug compounds, in accordance with an exemplary embodiment of the disclosure. Referring to FIG. 6, the hardware implementation shown by a representation 600 for the system 102 that employs a processing system 602 for selection of a set of candidate drug compounds, as described herein.

In some examples, the processing system 602 may comprise one or more hardware processor 604, a non-transitory computer-readable medium 606, a bus 608, a bus interface 610, and a transceiver 612. FIG. 6 further illustrates the set of interfaces 102a, the knowledge base 106, the knowledge processing engines 107, set of ontologies 108, the pathway generation engine 110, the search engine 112, the screening engine 114, the expression analysis engine 116, the ANS engine 118, the molecular stability analysis engine 120, the safety analysis engine 122, the network analysis engine 124, the clustering engine 126, and the drug selection engine 128, as described in detail in FIG. 1.

The hardware processor 604 may be configured to manage the bus 608 and general processing, including the execution of a set of instructions stored on the computer-readable medium 306. The set of instructions, when executed by the processor 304, causes the system 102 to execute the various functions described herein for any particular apparatus. The hardware processor 604 may be implemented, based on a number of processor technologies known in the art. Examples of the hardware processor 604 may be a Reduced Instruction Set Computing (RISC) processor, an Application-Specific Integrated Circuit (ASIC) processor, a Complex Instruction Set Computing (CISC) processor, and/or other processors or control circuits.

The non-transitory computer-readable medium 606 may be used for storing data that is manipulated by the hardware processor 604 when executing the set of instructions. The data is stored for short periods or in the presence of power. The computer-readable medium 306 may also be configured to store data for one or more of the set of interfaces 102a, the knowledge base 106, the knowledge processing engines 107, set of ontologies 108, the pathway generation engine 110, the search engine 112, the screening engine 114, the expression analysis engine 116, the ANS engine 118, the molecular stability analysis engine 120, the safety analysis engine 122, the network analysis engine 124, the clustering engine 126, and the drug selection engine 128.

The bus 608 is configured to link together various circuits. In this example, the system 102 employing the processing system 602 and the non-transitory computer-readable medium 606 may be implemented with bus architecture, represented generally by bus 608. The bus 608 may include any number of interconnecting buses and bridges depending on the specific implementation of the system 102 and the overall design constraints. The bus interface 610 may be configured to provide an interface between the bus 608 and other circuits, such as, the transceiver 612, and external devices, such as the data sources 104.

The transceiver 612 may be configured to provide a communication of the system 102 with various other apparatus, such as the data sources 104, via a network. The transceiver 612 may communicate via wireless communication with networks, such as the Internet, the Intranet and/or a wireless network, such as a cellular telephone network, a wireless local area network (WLAN) and/or a metropolitan area network (MAN). The wireless communication may use any of a plurality of communication standards, protocols and technologies, such as 5th generation mobile network, Global System for Mobile Communications (GSM), Enhanced Data GSM Environment (EDGE), Long Term Evolution (LTE), wideband code division multiple access (W-CDMA), code division multiple access (CDMA), time division multiple access (TDMA), Bluetooth, Wireless Fidelity (Wi-Fi) (such as IEEE 802.11a, IEEE 802.11b, IEEE 802.11g and/or IEEE 802.11n), voice over Internet Protocol (VoIP), and/or Wi-MAX.

It should be recognized that, in some embodiments of the disclosure, one or more components of FIG. 6 may include software whose corresponding code may be executed by at least one processor, for across multiple processing environments. For example, the set of interfaces 102a, the knowledge base 106, the knowledge processing engines 107, set of ontologies 108, the pathway generation engine 110, the search engine 112, the screening engine 114, the expression analysis engine 116, the ANS engine 118, the molecular stability analysis engine 120, the safety analysis engine 122, the network analysis engine 124, the clustering engine 126, and the drug selection engine 128 may include software that may be executed across a single or multiple processing environments.

In an aspect of the disclosure, the hardware processor 604, the non-transitory computer-readable medium 606, or a combination of both may be configured or otherwise specially programmed to execute the operations or functionality of the set of interfaces 102a, the knowledge base 106, the knowledge processing engines 107, set of ontologies 108, the pathway generation engine 110, the search engine 112, the screening engine 114, the expression analysis engine 116, the ANS engine 118, the molecular stability analysis engine 120, the safety analysis engine 122, the network analysis engine 124, the clustering engine 126, and the drug selection engine 128, or various other components described herein, as described with respect to FIGS. 1 to 5B.

Various embodiments of the disclosure comprise the system 102 that may be configured to select a set of candidate drug compounds. The system 102 may comprise, for example, the set of interfaces 102a, the knowledge base 106, the knowledge processing engines 107, set of ontologies 108, the pathway generation engine 110, the search engine 112, the screening engine 114, the expression analysis engine 116, the ANS engine 118, the molecular stability analysis engine 120, the safety analysis engine 122, the network analysis engine 124, the clustering engine 126.

Various embodiments of the disclosure comprise the system 102 that may be configured to select a set of candidate drug compounds. The pathway generation engine 110 may generate a plurality of knowledge-based pathways based on at least relevant information. The relevant information may be extracted from the structured information based on the ontology of interest. The pathway generation engine 110 may further identify a set of target structures based on the plurality of knowledge-based pathways. The ANS engine 118 may determine a plurality of candidate drug compounds for the identified set of target structures. The safety analysis engine 122 may select the set of candidate drug compounds from the plurality of candidate drug compounds based on safety analysis of the plurality of candidate drug compounds using the lethality index.

Various embodiments of the disclosure may provide a non-transitory computer-readable medium having stored thereon; computer implemented instruction that when executed by a processor causes the system 102 to select a set of candidate drug compounds. The system 102 may execute operations comprising generating a plurality of knowledge-based pathways based on at least relevant information. The relevant information is extracted from structured information based on an ontology of interest. The system 102 may execute operations comprising identifying a set of target structures based on the plurality of knowledge-based pathways, and determining a plurality of candidate drug compounds for the identified set of target structures. The system 102 may further execute operations comprising selecting a set of candidate drug compounds from the plurality of candidate drug compounds based on safety analysis of the plurality of candidate drug compounds using a lethality index.

As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” to perform a function whenever the circuitry comprises the necessary hardware and/or code (if any is necessary) to perform the function, regardless of whether performance of the function is disabled, or not enabled, by some user-configurable setting.

The terminology used herein is for the purpose of describing particular embodiments only and is not intended to be limiting of embodiments of the disclosure. As used herein, the singular forms “a”, “an” and “the” are intended to include the plural forms as well, unless the context clearly indicates otherwise. It will be further understood that the terms “comprises”, “comprising”, “includes” and/or “including”, when used herein, specify the presence of stated features, integers, steps, operations, elements, and/or components, but do not preclude the presence or addition of one or more other features, integers, steps, operations, elements, components, and/or groups thereof.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequences of actions described herein can be considered to be embodied entirely within any non-transitory form of computer readable storage medium having stored therein a corresponding set of computer instructions that upon execution would cause an associated processor to perform the functionality described herein. Thus, the various aspects of the disclosure may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

Another embodiment of the disclosure may provide a non-transitory machine and/or computer-readable storage and/or media, having stored thereon, a machine code and/or a computer program having at least one code section executable by a machine and/or a computer, thereby causing the machine and/or computer to perform the steps as described herein for selection of a set of candidate drug compounds.

The present disclosure may also be embedded in a computer program product, which comprises all the features enabling the implementation of the methods described herein, and which when loaded in a computer system is able to carry out these methods. Computer program in the present context means any expression, in any language, code or notation, either statically or dynamically defined, of a set of instructions intended to cause a system having an information processing capability to perform a particular function either directly or after either or both of the following: a) conversion to another language, code or notation; b) reproduction in a different material form.

Further, those of skill in the art will appreciate that the various illustrative logical blocks, modules, circuits, algorithms, and/or steps described in connection with the embodiments disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The methods, sequences and/or algorithms described in connection with the embodiments disclosed herein may be embodied directly in firmware, hardware, in a software module executed by a processor, or in a combination thereof. A software module may reside in RAM memory, flash memory, ROM memory, EPROM memory, EEPROM memory, registers, hard disk, physical and/or virtual disk, a removable disk, a CD-ROM, virtualized system or device such as a virtual server or container, or any other form of storage medium known in the art. An exemplary storage medium is communicatively coupled to the processor (including logic/code executing in the processor) such that the processor can read information from, and write information to, the storage medium. In the alternative, the storage medium may be integral to the processor.

While the present disclosure has been described with reference to certain embodiments, it will be noted understood by, for example, those skilled in the art that various changes and modifications could be made and equivalents may be substituted without departing from the scope of the present disclosure as defined, for example, in the appended claims. In addition, many modifications may be made to adapt a particular situation or material to the teachings of the present disclosure without departing from its scope. The functions, steps and/or actions of the method claims in accordance with the embodiments of the disclosure described herein need not be performed in any particular order. Furthermore, although elements of the disclosure may be described or claimed in the singular, the plural is contemplated unless limitation to the singular is explicitly stated. Therefore, it is intended that the present disclosure is not limited to the particular embodiment disclosed, but that the present disclosure will include all embodiments falling within the scope of the appended claims.

What is claimed is:

1. A method, comprising:
generating, by one or more processors, a plurality of knowledge-based pathways based on at least relevant information,
wherein the relevant information is extracted from structured information based on an ontology of interest;
identifying, by the one or more processors, a set of target structures based on the plurality of knowledge-based pathways;
determining, by the one or more processors, a plurality of candidate drug compounds for the identified set of target structures; and
selecting, by the one or more processors, a set of candidate drug compounds from the plurality of candidate drug compounds based on safety analysis of the plurality of candidate drug compounds using a lethality index.

2. The method according to claim 1, wherein the ontology of interest is a life science ontology that comprises a plurality of biomedical terms and a plurality of data connections, and
wherein the structured information comprises at least a number of principal investigators, intervention used in clinical trials, expressions, biological functions, mutations and mechanism of actions retrieved from the relevant publications and the clinical trial registries associated with a medical condition of the host entity.

3. The method according to claim 1, further comprising retrieving, by the one or more processors, unstructured data from data sources via interfaces and application program interfaces (APIs),
wherein the data sources store a repository of publications, clinical trials, congresses, patents, grants, drug profiles, and gene profiles.

4. The method according to claim 3, further comprising extracting, by the one or more processors, the structured information from the unstructured data based on one or more artificial intelligence and natural language processing techniques.

5. The method according to claim 1, further comprising performing, by the one or more processors, a computational docking-based virtual screening for prioritization of a first set of candidate drug compounds corresponding to the identified set of target structures based on one or more scores,
wherein the plurality of candidate drug compounds is determined based on the first set of candidate drug compounds.

6. The method according to claim 5, wherein a first score of the one or more scores is a quantitative docking score that corresponds to performance of each candidate drug compound for each target structure, and
wherein a second score of the one or more scores is an affinity score that corresponds to an overall strength of binding affinity of each candidate drug compound based on a spatial arrangement of docking pose and presence of hydrogen bond interactions with each target structure.

7. The method according to claim 1, further comprising determining, by the one or more processors, a second set of candidate drug compounds based on a plurality of direct and in-direct connections between a plurality of biological entities in a biological network and the ontology of interest,
wherein the plurality of candidate drug compounds is determined based on the second set of candidate drug compounds.

8. The method according to claim 1, further comprising determining, by the one or more processors, a third set of candidate drug compounds based on a first analysis and a second analysis,
wherein the first analysis is associated with gene and protein expression profile of the identified set of target structures,
wherein the second analysis is associated with expression profiles of the third set of candidate drug compounds and corresponding pharmacokinetics effect, and
wherein the plurality of candidate drug compounds is determined based on the third set of candidate drug compounds.

9. The method according to claim 1, further comprising:
normalizing, by the one or more processors, the plurality of candidate drug compounds based on cross-mapping through the ontology of interest; and
scoring, by the one or more processors, the plurality of candidate drug compounds based on one or more parameters.

10. The method according to claim 9, further comprising performing, by one or more processors, molecular dynamics simulation on the plurality of candidate drug compounds to identify interaction stability with the identified set of target structures.

11. The method according to claim 1, wherein the lethality index corresponds to a scatter plot with safety coordinates which positions adverse events on a universal lethality index versus a universal frequency index.

12. The method according to claim 1, further comprising:
determining, by the one or more processors, prioritized target structures based on a mapping of the set of target structures and a list of target structures,
wherein the list of target structures is associated with a gene ontology corresponding to a host viral interaction;
identifying, by the one or more processors, a plurality of data connections, corresponding to the prioritized target structures, from the plurality of biological networks;
determining, by the one or more processors, a target connection network corresponding to the identified plurality of data connections;
detecting, by the one or more processors, a plurality of clusters corresponding to the plurality of data connections in the target connection network based on a graph-embedded self-clustering technique; and
determining, by the one or more processors, at least a first drug combination of at least a first candidate drug compound and a second candidate drug compound based on a combination score,
wherein the first candidate drug compound corresponds to a first cluster and the second candidate drug compound corresponds to a second cluster.

13. The method according to claim 12, further comprising mapping, by the one or more processors, each of the plurality of candidate drug compounds with a target structure of each cluster.

14. The method according to claim 12, further comprising calculating, by the one or more processors, the combination score for at least the first drug combination based on at least docking scores, lethality scores and safety scores corresponding to the first candidate drug compound and the second candidate drug compound, and
wherein the combination score for at least the first drug combination exceeds a threshold value.

15. The method according to claim 14, further comprising determining, by the one or more processors, a rank of the first drug combination based on a corresponding percentile score with respect to other drug combinations.

16. A system, comprising:
one or more processors configured to:
generate a plurality of knowledge-based pathways based on at least relevant information,
wherein the relevant information is extracted from structured information based on an ontology of interest:
identify a set of target structures based on the plurality of knowledge-based pathways;
determine a plurality of candidate drug compounds for the identified set of target structures; and
select a set of candidate drug compounds from the plurality of candidate drug compounds based on safety analysis of the plurality of candidate drug compounds using a lethality index.

17. The system according to claim 16, wherein the lethality index corresponds to a scatter plot with safety coordinates which positions adverse events on a universal lethality index versus a universal frequency index.

18. The system according to claim 16, wherein the one or more processors are further configured to:
determine prioritized target structures based on a mapping of the set of target structures and a list of target structures,
wherein the list of target structures is associated with a gene ontology corresponding to a host viral interaction;
identify a plurality of data connections, corresponding to the prioritized target structures, from the plurality of biological networks;
determine a target connection network corresponding to the identified plurality of data connections;
detect a plurality of clusters corresponding to the plurality of data connections in the target connection network based on a graph-embedded self-clustering technique; and
determine at least a first drug combination of at least a first candidate drug compound and a second candidate drug compound based on a combination score,
wherein the first candidate drug compound corresponds to a first cluster and the second candidate drug compound corresponds to a second cluster.

19. The system according to claim 18, wherein the one or more processors are further configured to calculate the combination score for at least the first drug combination based on at least docking scores, lethality scores and safety scores corresponding to the first candidate drug compound and the second candidate drug compound, and
wherein the combination score for at least the first drug combination exceeds a threshold value.

20. A non-transitory computer-readable medium having stored thereon, computer implemented instruction that when executed by a processor in a computer, causes the computer to execute operations, the operations comprising:
generating a plurality of knowledge-based pathways based on at least relevant information,
wherein the relevant information is extracted from structured information based on an ontology of interest;
identifying a set of target structures based on the plurality of knowledge-based pathways;
determining a plurality of candidate drug compounds for the identified set of target structures; and
selecting a set of candidate drug compounds from the plurality of candidate drug compounds based on safety analysis of the plurality of candidate drug compounds using a lethality index.
